SEQUENCE LISTING (89 sequences)

SEQ ID NO: 1 - protein, 13 aa, herpes simplex virus 7
DALDDFDLDMLGS

SEQ ID NO: 2 - protein, 55 aa, Artificial Sequence
  Synthetic construct
GRADALDDFDLDMLGSDALDDFDLDMLGSDALDDFDLDMLGSDALDDFDLDMLIN

SEQ ID NO: 3 - protein, 102 aa, Homo sapiens
KGFGAFERSILTQIDHILMDKERLLRRTQTKRSVYRVLGKPEPAAQPVPESLPGEPEILPQAPANAHLKDLDEEIFDDDDFYHQLLRELIERKTSSLDPNDQ

SEQ ID NO: 4 - protein, 31 aa, Homo sapiens
PGLPNGLLSGDEDFSSIADMDFSALLSQISS

SEQ ID NO: 5 - protein, 48 aa, Homo sapiens
PYTPNLPHHQNGHLQHHPPMPPHPGHYWPVHNELAFQPPISNHPAPEY

SEQ ID NO: 6 - protein, 100 aa, Homo sapiens
PPHLNPQDPLKDLVSLACDPASQQPGPLNGSGQLKMPSHCLSAQMLAPPPPGLPRLALPPATKPATTSEGGATSPTSPSYSPPDTSPANRSFVGLGPRDP

SEQ ID NO: 7 - protein, 68 aa, Homo sapiens
ADFQPPYFPPPYQPIYPQSQDPYSHVNDPYSLNPLHAQPQPQHPGWPGQRQSQESGLLHTHRGLPHQL

SEQ ID NO: 8 - protein, 112 aa, Homo sapiens
NRTVSGGQYVVAAAPNLQNQQVLTGLPGVMPNIQYQVIPQFQTVDGQQLQFAATGAQVQQDGSGQIQIIPGANQQIITNRGSGGNIIAAMPNLLQQAVPLQGLANNVLSGQT

SEQ ID NO: 9 - protein, 143 aa, Homo sapiens
QGQTPQRVSGLQGSDALNIQQNQTSGGSLQAGQQKEGEQNQQTQQQQILIQPQLVQGGQALQALQAAPLSGQTFTTQAISQETLQNLQLQAVPNSGPIIIRTPTVGPNGQVSWQTLQLQNLQVQNPQAQTITLAPMQGVSLGQ

SEQ ID NO: 10 - protein, 95 aa, Homo sapiens
DLQQLQQLQQQNLNLQQFVLVHPTTNLQPAQFIISQTPQGQQGLLQAQNLLTQLPQQSQANLLQSQPSITLTSQPATPTRTIAATPIQTLPQSQS

SEQ ID NO: 11 - protein, 63 aa, Homo sapiens
QLAGDIQQLLQLQQLVLVPGHHLQPPAQFLLPQAQQSQPGLLPTPNLFQLPQQTQGALLTSQP

SEQ ID NO: 12 - protein, 90 aa, Artificial Sequence
  synthetic construct
NLFQLPQQTQGALLTSQPNLFQLPQQTQGALLTSQPNLFQLPQQTQGALLTSQPNLFQLPQQTQGALLTSQPNLFQLPQQTQGALLTSQP

SEQ ID NO: 13 - protein, 91 aa, Homo sapiens
PPSTGNSASLSLPLVLQPGLSEPPQPLLPASAPSAPPPAPSLGPGSQQAAFGNPPALLQPPEVPVPHSTQFAANHQEFLPHPQAPQPIVPG

SEQ ID NO: 14 - protein, 111 aa, Homo sapiens
MATRVLSMSARLGPVPQPPAPQDEPVFAQLKPVLGAANPARDAALFPGEELKHAHHRPQAQPAPAQAPQPAQPPATGPRLPPEDLVQTRCEMEKYLTPQLPPVPIIPEHKK

SEQ ID NO: 15 - protein, 88 aa, Homo sapiens
MALSEPILPSFSTFASPCRERGLQERWPRAEPESGGTDDDLNSVLDFILSMGLDGLGAEAAPEPPPPPPPPAFYYPEPGAPPPYSAPA

SEQ ID NO: 16 - protein, 11 aa, Human immunodeficiency virus
YGRKKRRQRRR

SEQ ID NO: 17 - DNA, 1000 bp, Homo sapiens
gaaatttgggaggggagccatcaaagaagcctgggagcagcagttccagggaaaaaggagaatgtgatggccagagagccaaaagaaaaagtagttgaaggagtgctcagcactaggcatctgaactgaatgctgtggcaggctcactggccacaaacaatagggagctggtggaggccttgacgaggaccatttcaacaaactggtgggcttaaaatccggaagaaacagttgaacaaatcattttgacgccttttataaaccacacaagcttattccaaacccgttactggcctaactgatttaagtccctttcccatctgatcctcagagattctaagggacttagcctatccatgactcttcgtcctgcttctcacctcccatgattgccctaacgatgtgaaagtgctttcaaacaaagatgcccaagaaagaaggtaggcaaatgtgcaagcattagtttgtagtacgctattactgtatttcaccttgcactctctagtttccttcgtgctccctcaatatccaactcttaataaattcatggctcccggtgagcattcatcaattctcattccacgcctttagcccttcccgttcccgcccaactctcgctccctcccctggccaaatctctaacctgcaaggctaattccgaattccaaatcggaagcaagagggcggggccccgtgagaggcgatggattgctccagtccgttcccgacgcactgtgcgcatgcgctggtcctccgcggaccgttcgtgctgcccgcctagaaagggtgaagtggttgtttccgtgacggactgagtacgggtgcctgtcaggctcttgcggaagtccatgcgccattgggagggcctcggccgcggctctgtgcccttgctgctgagggccacttcctgggtcattcctggaccgggagccgggctggggctcacacgggggctcccgcgtggccgtctcggcgcctgcgtgacctccccgccggcggg

SEQ ID NO: 18 - protein, 12 aa, Artificial Sequence
  Synthetic construct
PVRRPRRRRRRK

SEQ ID NO: 19 - protein, 12 aa, Artificial Sequence
  Synthetic construct
THRLPRRRRRRK

SEQ ID NO: 20 - protein, 9 aa, Artificial Sequence
  Synthetic construct
RRRRRRRRR

SEQ ID NO: 21 - protein, 16 aa, Drosophila melanogaster
RQILIWFQNRRMKWKK

SEQ ID NO: 22 - DNA, 18 bp, Homo sapiens
gaaaaagtagttgaagga

SEQ ID NO: 23 - DNA, 18 bp, Homo sapiens
gtagttgaaggagtgctc

SEQ ID NO: 24 - DNA, 18 bp, Homo sapiens
gacctccccgccggcggg

SEQ ID NO: 25 - DNA, 20 bp, Homo sapiens
ctcttgcggaagtccatgcg

SEQ ID NO: 26 - protein, 168 aa, Artificial Sequence
  synthetic construct
GEKPYKCPECGKSFSQRAHLERHQRTHTGEKPYKCPECGKSFSQSSNLVRHQRTHTGEKPYKCPECGKSFSTSGSLVRHQRTHTGEKPYKCPECGKSFSQSSSLVRHQRTHTGEKPYKCPECGKSFSQRANLRAHQRTHTGEKPYKCPECGKSFSQSSNLVRHQRTHT

SEQ ID NO: 27 - protein, 168 aa, Artificial Sequence
  synthetic construct
GEKPYKCPECGKSFSSKKHLAEHQRTHTGEKPYKCPECGKSFSQSSSLVRHQRTHTGEKPYKCPECGKSFSDPGHLVRHQRTHTGEKPYKCPECGKSFSQRAHLERHQRTHTGEKPYKCPECGKSFSTSGSLVRHQRTHTGEKPYKCPECGKSFSQSSSLVRHQRTHT

SEQ ID NO: 28 - protein, 168 aa, Artificial Sequence
  synthetic construct
GEKPYKCPECGKSFSRNDTLTEHQRTHTGEKPYKCPECGKSFSTSGSLVRHQRTHTGEKPYKCPECGKSFSDPGHLVRHQRTHTGEKPYKCPECGKSFSQRAHLERHQRTHTGEKPYKCPECGKSFSTSGSLVRHQRTHTGEKPYKCPECGKSFSQSSSLVRHQRTHT

SEQ ID NO: 29 - protein, 168 aa, Artificial Sequence
  synthetic construct
GEKPYKCPECGKSFSSKKALTEHQRTHTGEKPYKCPECGKSFSDPGHLVRHQRTHTGEKPYKCPECGKSFSRSDNLVRHQRTHTGEKPYKCPECGKSFSRSDNLVRHQRTHTGEKPYKCPECGKSFSQSSNLVRHQRTHTGEKPYKCPECGKSFSDPGHLVRHQRTHT

SEQ ID NO: 30 - protein, 168 aa, Artificial Sequence
  synthetic construct
GEKPYKCPECGKSFSSKKHLAEHQRTHTGEKPYKCPECGKSFSTSGSLVRHQRTHTGEKPYKCPECGKSFSQRAHLERHQRTHTGEKPYKCPECGKSFSQSSNLVRHQRTHTGEKPYKCPECGKSFSDPGALVRHQRTHTGEKPYKCPECGKSFSTSGSLVRHQRTHT

SEQ ID NO: 31 - protein, 168 aa, Artificial Sequence
  synthetic construct
GEKPYKCPECGKSFSDPGALVRHQRTHTGEKPYKCPECGKSFSRSDNLVRHQRTHTGEKPYKCPECGKSFSRSDKLVRHQRTHTGEKPYKCPECGKSFSDPGHLVRHQRTHTGEKPYKCPECGKSFSDCRDLARHQRTHTGEKPYKCPECGKSFSSKKHLAEHQRTHT

SEQ ID NO: 32 - protein, 168 aa, Artificial Sequence
  synthetic construct
GEKPYKCPECGKSFSRSDELVRHQRTHTGEKPYKCPECGKSFSRSDELVRHQRTHTGEKPYKCPECGKSFSTSGNLVRHQRTHTGEKPYKCPECGKSFSRSDKLTEHQRTHTGEKPYKCPECGKSFSTSGNLTEHQRTHTGEKPYKCPECGKSFSDPGHLVRHQRTHT

SEQ ID NO: 33 - protein, 168 aa, Artificial Sequence
  synthetic construct
GEKPYKCPECGKSFSRSDELVRHQRTHTGEKPYKCPECGKSFSRSDELVRHQRTHTGEKPYKCPECGKSFSTSGNLVRHQRTHTGEKPYKCPECGKSFSRSDKLTEHQRTHTGEKPYKCPECGKSFSTSGNLTEHQRTHTGEKPYKCPECGKSFSQSSSLVRHQRTHT

SEQ ID NO: 34 - protein, 168 aa, Artificial Sequence
  synthetic construct
GEKPYKCPECGKSFSRSDELVRHQRTHTGEKPYKCPECGKSFSRSDELVRHQRTHTGEKPYKCPECGKSFSTSGNLVRHQRTHTGEKPYKCPECGKSFSRSDKLTEHQRTHTGEKPYKCPECGKSFSTSGNLTEHQRTHTGEKPYKCPECGKSFSDCRDLARHQRTHT

SEQ ID NO: 35 - protein, 168 aa, Artificial Sequence
  synthetic construct
GEKPYKCPECGKSFSRSDELVRHQRTHTGEKPYKCPECGKSFSRSDELVRHQRTHTGEKPYKCPECGKSFSTSGNLVRHQRTHTGEKPYKCPECGKSFSRSDKLTEHQRTHTGEKPYKCPECGKSFSTSGNLTEHQRTHTGEKPYKCPECGKSFSRSDELVRHQRTHT

SEQ ID NO: 36 - protein, 168 aa, Artificial Sequence
  synthetic construct
GEKPYKCPECGKSFSDPGHLVRHQRTHTGEKPYKCPECGKSFSSRRTCRAHQRTHTGEKPYKCPECGKSFSTSGELVRHQRTHTGEKPYKCPECGKSFSQSGDLRRHQRTHTGEKPYKCPECGKSFSRSDNLVRHQRTHTGEKPYKCPECGKSFSSRRTCRAHQRTHT

SEQ ID NO: 37 - protein, 168 aa, Artificial Sequence
  synthetic construct
GEKPYKCPECGKSFSQSSSLVRHQRTHTGEKPYKCPECGKSFSTSGNLTEHQRTHTGEKPYKCPECGKSFSQSSSLVRHQRTHTGEKPYKCPECGKSFSQSSNLVRHQRTHTGEKPYKCPECGKSFSRSDDLVRHQRTHTGEKPYKCPECGKSFSQSGNLTEHQRTHT

SEQ ID NO: 38 - protein, 168 aa, Artificial Sequence
  synthetic construct
GEKPYKCPECGKSFSDPGHLVRHQRTHTGEKPYKCPECGKSFSRNDALTEHQRTHTGEKPYKCPECGKSFSTSGSLVRHQRTHTGEKPYKCPECGKSFSQSGDLRRHQRTHTGEKPYKCPECGKSFSRSDKLVRHQRTHTGEKPYKCPECGKSFSSKKHLAEHQRTHT

SEQ ID NO: 39 - protein, 168 aa, Artificial Sequence
  synthetic construct
GEKPYKCPECGKSFSDPGHLVRHQRTHTGEKPYKCPECGKSFSQSGNLTEHQRTHTGEKPYKCPECGKSFSQSSSLVRHQRTHTGEKPYKCPECGKSFSQSSNLVRHQRTHTGEKPYKCPECGKSFSRSDDLVRHQRTHTGEKPYKCPECGKSFSSKKHLAEHQRTHT

SEQ ID NO: 40 - protein, 168 aa, Artificial Sequence
  synthetic construct
GEKPYKCPECGKSFSDPGHLVRHQRTHTGEKPYKCPECGKSFSRNDALTEHQRTHTGEKPYKCPECGKSFSTSGSLVRHQRTHTGEKPYKCPECGKSFSQSGDLRRHQRTHTGEKPYKCPECGKSFSRSDKLVRHQRTHTGEKPYKCPECGKSFSTTGALTEHQRTHT

SEQ ID NO: 41 - protein, 168 aa, Artificial Sequence
  synthetic construct
GEKPYKCPECGKSFSDPGHLVRHQRTHTGEKPYKCPECGKSFSSRRTCRAHQRTHTGEKPYKCPECGKSFSQRAHLERHQRTHTGEKPYKCPECGKSFSDCRDLARHQRTHTGEKPYKCPECGKSFSQSGDLRRHQRTHTGEKPYKCPECGKSFSTKNSLTEHQRTHT

SEQ ID NO: 42 - protein, 168 aa, Artificial Sequence
  synthetic construct
GEKPYKCPECGKSFSRSDNLVRHQRTHTGEKPYKCPECGKSFSTTGALTEHQRTHTGEKPYKCPECGKSFSRSDELVRHQRTHTGEKPYKCPECGKSFSQSGDLRRHQRTHTGEKPYKCPECGKSFSDPGNLVRHQRTHTGEKPYKCPECGKSFSTKNSLTEHQRTHT

SEQ ID NO: 43 - protein, 168 aa, Artificial Sequence
  synthetic construct
GEKPYKCPECGKSFSRSDDLVRHQRTHTGEKPYKCPECGKSFSSRRTCRAHQRTHTGEKPYKCPECGKSFSRSDDLVRHQRTHTGEKPYKCPECGKSFSTSGSLVRHQRTHTGEKPYKCPECGKSFSTSGELVRHQRTHTGEKPYKCPECGKSFSQNSTLTEHQRTHT

SEQ ID NO: 44 - protein, 276 aa, Artificial Sequence
  synthetic construct
MPKKKRKVGLEPGEKPYKCPECGKSFSQRAHLERHQRTHTGEKPYKCPECGKSFSQSSNLVRHQRTHTGEKPYKCPECGKSFSTSGSLVRHQRTHTGEKPYKCPECGKSFSQSSSLVRHQRTHTGEKPYKCPECGKSFSQRANLRAHQRTHTGEKPYKCPECGKSFSQSSNLVRHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 45 - protein, 276 aa, Artificial Sequence
  synthetic construct
MPKKKRKVGLEPGEKPYKCPECGKSFSSKKHLAEHQRTHTGEKPYKCPECGKSFSQSSSLVRHQRTHTGEKPYKCPECGKSFSDPGHLVRHQRTHTGEKPYKCPECGKSFSQRAHLERHQRTHTGEKPYKCPECGKSFSTSGSLVRHQRTHTGEKPYKCPECGKSFSQSSSLVRHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 46 - protein, 276 aa, Artificial Sequence
  synthetic construct
MPKKKRKVGLEPGEKPYKCPECGKSFSRNDTLTEHQRTHTGEKPYKCPECGKSFSTSGSLVRHQRTHTGEKPYKCPECGKSFSDPGHLVRHQRTHTGEKPYKCPECGKSFSQRAHLERHQRTHTGEKPYKCPECGKSFSTSGSLVRHQRTHTGEKPYKCPECGKSFSQSSSLVRHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 47 - protein, 276 aa, Artificial Sequence
  synthetic construct
MPKKKRKVGLEPGEKPYKCPECGKSFSSKKALTEHQRTHTGEKPYKCPECGKSFSDPGHLVRHQRTHTGEKPYKCPECGKSFSRSDNLVRHQRTHTGEKPYKCPECGKSFSRSDNLVRHQRTHTGEKPYKCPECGKSFSQSSNLVRHQRTHTGEKPYKCPECGKSFSDPGHLVRHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 48 - protein, 276 aa, Artificial Sequence
  synthetic construct
MPKKKRKVGLEPGEKPYKCPECGKSFSSKKHLAEHQRTHTGEKPYKCPECGKSFSTSGSLVRHQRTHTGEKPYKCPECGKSFSQRAHLERHQRTHTGEKPYKCPECGKSFSQSSNLVRHQRTHTGEKPYKCPECGKSFSDPGALVRHQRTHTGEKPYKCPECGKSFSTSGSLVRHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 49 - protein, 276 aa, Artificial Sequence
  synthetic construct
MPKKKRKVGLEPGEKPYKCPECGKSFSDPGALVRHQRTHTGEKPYKCPECGKSFSRSDNLVRHQRTHTGEKPYKCPECGKSFSRSDKLVRHQRTHTGEKPYKCPECGKSFSDPGHLVRHQRTHTGEKPYKCPECGKSFSDCRDLARHQRTHTGEKPYKCPECGKSFSSKKHLAEHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 50 - protein, 276 aa, Artificial Sequence
  synthetic construct
MPKKKRKVGLEPGEKPYKCPECGKSFSRSDELVRHQRTHTGEKPYKCPECGKSFSRSDELVRHQRTHTGEKPYKCPECGKSFSTSGNLVRHQRTHTGEKPYKCPECGKSFSRSDKLTEHQRTHTGEKPYKCPECGKSFSTSGNLTEHQRTHTGEKPYKCPECGKSFSDPGHLVRHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 51 - protein, 276 aa, Artificial Sequence
  synthetic construct
MPKKKRKVGLEPGEKPYKCPECGKSFSRSDELVRHQRTHTGEKPYKCPECGKSFSRSDELVRHQRTHTGEKPYKCPECGKSFSTSGNLVRHQRTHTGEKPYKCPECGKSFSRSDKLTEHQRTHTGEKPYKCPECGKSFSTSGNLTEHQRTHTGEKPYKCPECGKSFSQSSSLVRHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 52 - protein, 276 aa, Artificial Sequence
  synthetic construct
MPKKKRKVGLEPGEKPYKCPECGKSFSRSDELVRHQRTHTGEKPYKCPECGKSFSRSDELVRHQRTHTGEKPYKCPECGKSFSTSGNLVRHQRTHTGEKPYKCPECGKSFSRSDKLTEHQRTHTGEKPYKCPECGKSFSTSGNLTEHQRTHTGEKPYKCPECGKSFSDCRDLARHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 53 - protein, 276 aa, Artificial Sequence
  synthetic construct
MPKKKRKVGLEPGEKPYKCPECGKSFSRSDELVRHQRTHTGEKPYKCPECGKSFSRSDELVRHQRTHTGEKPYKCPECGKSFSTSGNLVRHQRTHTGEKPYKCPECGKSFSRSDKLTEHQRTHTGEKPYKCPECGKSFSTSGNLTEHQRTHTGEKPYKCPECGKSFSRSDELVRHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 54 - protein, 276 aa, Artificial Sequence
  synthetic construct
MPKKKRKVGLEPGEKPYKCPECGKSFSDPGHLVRHQRTHTGEKPYKCPECGKSFSSRRTCRAHQRTHTGEKPYKCPECGKSFSTSGELVRHQRTHTGEKPYKCPECGKSFSQSGDLRRHQRTHTGEKPYKCPECGKSFSRSDNLVRHQRTHTGEKPYKCPECGKSFSSRRTCRAHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 55 - protein, 276 aa, Artificial Sequence
  synthetic construct
MPKKKRKVGLEPGEKPYKCPECGKSFSQSSSLVRHQRTHTGEKPYKCPECGKSFSTSGNLTEHQRTHTGEKPYKCPECGKSFSQSSSLVRHQRTHTGEKPYKCPECGKSFSQSSNLVRHQRTHTGEKPYKCPECGKSFSRSDDLVRHQRTHTGEKPYKCPECGKSFSQSGNLTEHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 56 - protein, 276 aa, Artificial Sequence
  synthetic construct
MPKKKRKVGLEPGEKPYKCPECGKSFSDPGHLVRHQRTHTGEKPYKCPECGKSFSRNDALTEHQRTHTGEKPYKCPECGKSFSTSGSLVRHQRTHTGEKPYKCPECGKSFSQSGDLRRHQRTHTGEKPYKCPECGKSFSRSDKLVRHQRTHTGEKPYKCPECGKSFSSKKHLAEHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 57 - protein, 276 aa, Artificial Sequence
  synthetic construct
MPKKKRKVGLEPGEKPYKCPECGKSFSDPGHLVRHQRTHTGEKPYKCPECGKSFSQSGNLTEHQRTHTGEKPYKCPECGKSFSQSSSLVRHQRTHTGEKPYKCPECGKSFSQSSNLVRHQRTHTGEKPYKCPECGKSFSRSDDLVRHQRTHTGEKPYKCPECGKSFSSKKHLAEHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 58 - protein, 276 aa, Artificial Sequence
  synthetic construct
MPKKKRKVGLEPGEKPYKCPECGKSFSDPGHLVRHQRTHTGEKPYKCPECGKSFSRNDALTEHQRTHTGEKPYKCPECGKSFSTSGSLVRHQRTHTGEKPYKCPECGKSFSQSGDLRRHQRTHTGEKPYKCPECGKSFSRSDKLVRHQRTHTGEKPYKCPECGKSFSTTGALTEHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 59 - protein, 276 aa, Artificial Sequence
  synthetic construct
MPKKKRKVGLEPGEKPYKCPECGKSFSDPGHLVRHQRTHTGEKPYKCPECGKSFSSRRTCRAHQRTHTGEKPYKCPECGKSFSQRAHLERHQRTHTGEKPYKCPECGKSFSDCRDLARHQRTHTGEKPYKCPECGKSFSQSGDLRRHQRTHTGEKPYKCPECGKSFSTKNSLTEHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 60 - protein, 276 aa, Artificial Sequence
  synthetic construct
MPKKKRKVGLEPGEKPYKCPECGKSFSRSDNLVRHQRTHTGEKPYKCPECGKSFSTTGALTEHQRTHTGEKPYKCPECGKSFSRSDELVRHQRTHTGEKPYKCPECGKSFSQSGDLRRHQRTHTGEKPYKCPECGKSFSDPGNLVRHQRTHTGEKPYKCPECGKSFSTKNSLTEHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 61 - protein, 276 aa, Artificial Sequence
  synthetic construct
MPKKKRKVGLEPGEKPYKCPECGKSFSRSDDLVRHQRTHTGEKPYKCPECGKSFSSRRTCRAHQRTHTGEKPYKCPECGKSFSRSDDLVRHQRTHTGEKPYKCPECGKSFSTSGSLVRHQRTHTGEKPYKCPECGKSFSTSGELVRHQRTHTGEKPYKCPECGKSFSQNSTLTEHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 62 - protein, 7 aa, Simian virus 40
PKKKRKV

SEQ ID NO: 63 - protein, 6 aa, Artificial Sequence
  Synthetic construct
GGSGGS

SEQ ID NO: 64 - DNA, 4513 bp, Artificial Sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccgtatacctcgagcccggggaaaagccatataaatgccccgagtgcggcaaatcattcagccaaagtagcaacttagtaagacaccagcgcacccataccggtaagaaaactagtcttaagctcgagcccggggaaaaaccctataaatgccccgagtgtggtaagtcattctctcaaagcggggatttaagaagacaccagagaacccacaccggtaagaaaactagtggcgcgccctcgagcccggggagaaaccttataaatgcccagaatgcgggaaatcgttcagtcaaagagcacatttagaaagacatcaacggacccacaccggtaagaaaactagtcctaggctcgagcccggggaaaaaccttacaagtgccctgagtgcggcaagagcttctctcaatcaagttcattagtaagacaccagaggactcataccggtaagaaaactagtcctcagcctcgagcccggggagaagccttataagtgccctgagtgtggcaaaagcttcagcgatcctggaaatttagtaagacaccaacgcacccacaccggtaagaaaactagtatgcatctcgagcccggggaaaaaccgtataaatgtcctgagtgcggtaagtctttttccgactgtagagacttagcgagacaccaacgtactcataccggtaaaaagactagttgtacactcgagcccggggaaaaaccgtacaagtgtcctgagtgcgggaagagtttctccgatccgggccacttagtaagacatcagaggacacataccggtaaaaagactagtttcgaactcgagcccggggagaaaccatacaaatgccccgagtgtggaaagtcatttagtgatccaggcgcattagtaagacatcagcggacacataccggtaagaaaactagtgaattcctcgagcccggggagaagccatataaatgtcccgagtgtggcaagtccttttctagatcagataatttagtaagacatcagagaacgcacaccggtaaaaagactagtcaattgctcgagcccggggagaagccatacaagtgtcccgaatgcgggaagtcattctccagaagtgacgatttagtaagacatcagcgcacgcacaccggtaagaaaactagtccatggctcgagcccggggagaagccctacaagtgtccagaatgcggaaagagtttctccagaagtgacaaattagtaagacaccagagaacccataccggtaagaaaactagtcatatgctcgagcccggggagaagccgtacaagtgccctgaatgtggtaagtcattttcgagaagtgatgaattagtaagacaccagcggactcataccggtaaaaagactagtgctagcctcgagcccggggagaagccctataaatgtccagaatgtggaaagtcctttagcacgtcagggaacttagtaagacaccagcgaactcataccggtaagaaaactagtttaattaactcgagcccggggagaaaccatacaagtgtccagagtgcgggaaaagctttagtacaagcggtgagttagtaagacaccaacgaacacacaccggtaaaaagactagtgtttaaacctcgagcccggggaaaagccctacaagtgcccggaatgcggcaagtcttttagcaccagcggacatttagtaagacaccagagaacccacaccggtaaaaagactagtccgcggctcgagcccggggaaaagccctacaagtgtcctgagtgcggaaagtctttctccactagcggttcattagtaagacaccagaggacacacaccggtaaaaagactagtgcatgcgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 65 - DNA, 4442 bp, Artificial Sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgtatacctcgagcccggggagaagccctataaatgccctgaatgcgggaaatctttctcttctaagaaggcactcacagaacaccagcggacacacaccggtaaaaaaactagtcttaagctcgagcccggggaaaagccctacaagtgccccgaatgcgggaagtcttttagtcagagtggaaatcttaccgagcaccagagaacacacaccggtaagaagactagtggcgcgccctcgagcccggggagaagccatacaagtgccctgaatgtggcaagtccttttcaagagccgataacctgacagaacaccaaaggacgcataccggtaagaaaactagtcctaggctcgagcccggggagaagccctataaatgccctgaatgtggcaagagcttcagtactagcgggaatctcactgaacatcagcgaactcataccggtaaaaaaactagtcctcagcctcgagcccggggaaaaaccatacaagtgccctgagtgcggcaagagttttagtacctcacactctcttacagaacatcagcgaacccacaccggtaaaaaaactagtatgcatctcgagcccggggagaaaccatacaaatgtcccgaatgtggcaagagtttcagcagtaaaaagcatctcgctgagcatcagagaactcacaccggtaaaaagactagttgtacactcgagcccggggaaaagccctacaaatgccccgaatgtggtaagtctttttctaggaacgacaccttgacagaacaccagcggacccacaccggtaagaagactagtgaattcctcgagcccggggagaagccttataagtgccccgaatgtggaaagagtttctctactaagaatagcctgaccgagcaccagcgcactcacaccggtaagaaaactagtcaattgctcgagcccggggagaagccctataaatgccctgaatgcgggaaatctttctctcaatcaggccacctcacagaacaccagcggacacacaccggtaaaaaaactagtccatggctcgagcccggggagaaaccctataagtgtcccgaatgcgggaaatcattctctcatacagggcatctgctcgaacatcaaaggacgcacaccggtaaaaagactagtcatatgctcgagcccggggaaaagccttacaaatgccccgaatgtgggaagagtttcagccggtctgataagctgaccgaacaccagagaactcataccggtaaaaaaactagtgctagcctcgagcccggggaaaagccctacaagtgccctgagtgtgggaagtccttttcttcaagacgcacgtgccgcgctcaccagcggacacataccggtaagaaaactagtttaattaactcgagcccggggagaaaccatacaaatgtcccgaatgtggcaagtccttctcacagaactctactttgaccgagcatcagagaactcacaccggtaagaagactagtccgcggctcgagcccggggaaaagccttataagtgccccgaatgcggaaagagcttctcaaggaatgatgcacttaccgagcatcaaaggactcataccggtaaaaaaactagtgcatgcttcgaactcgagcccggggaaaagccctataagtgtcccgaatgcggcaagagttttagtactactggcgcactcacagaacaccagcgcactcacaccggtaagaaaactagtgaaagtcctctccactgactgtagcctccaattcactggagatctgacacaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 66 - DNA, 4376 bp, Artificial Sequence
  synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccgtatacctcgagcccggggagaagccatacaaatgccctgagtgtggaaagtcatttagccagcgagctaatctgcgggcccaccagcggacccacaccggtaagaagactagtcttaagctcgagcccggggagaagccatacaaatgtccagaatgtggaaagtccttctctgatagtggcaacctcagagtgcatcagcgaacacataccggtaagaagactagtggcgcgccctcgagcccggggaaaagccatataagtgccctgagtgtggaaagagcttcagtaggaaggataaccttaaaaaccaccaaagaacccacaccggtaagaagactagtcctaggctcgagcccggggaaaagccatataaatgtcccgagtgcggcaaatccttctctaccactggcaacctcacagtgcatcaacggactcacaccggtaaaaagactagtcctcagcctcgagcccggggaaaagccctataaatgtcccgagtgcggaaagtctttttccagccctgccgacctgacacgccaccaacgaacgcacaccggtaagaagactagtatgcatctcgagcccggggaaaagccgtacaaatgtccagagtgtggaaaatccttttctgataaaaaggacctgacacggcatcagcgaacccacaccggtaaaaagactagttgtacactcgagcccggggagaaaccttataaatgcccagaatgcggtaaaagtttcagcaggacggataccttgcgggatcatcagagaacccacaccggtaaaaaaactagtgaattcctcgagcccggggaaaaaccatacaagtgccccgagtgtggcaagagctttagtacccacctcgacctgattagacaccagcgcacccacaccggtaagaaaactagtcaattgctcgagcccggggaaaagccctataagtgcccagagtgcgggaaatcattctcacagctggcacatcttagagcccaccagcggacccacaccggtaagaagactagtccatggctcgagcccggggagaaaccctataagtgccctgaatgcggcaagtctttcagtgagcggtcacatctccgagagcaccagcgaacgcacaccggtaaaaagactagtcatatgctcgagcccggggaaaaaccctacaagtgccctgagtgtggaaagtcatttagtcgctccgaccacctgaccaaccatcagcggactcacaccggtaagaaaactagtgctagcctcgagcccggggagaaaccttacaagtgccccgagtgcggcaagagtttcagccacaggaccaccctgacaaaccaccagaggacccacaccggtaaaaagactagtttaattaactcgagcccggggagaaaccttataagtgtcctgagtgcggcaaaagtttctctcaaaagtcctcccttattgcccatcaaaggacccataccggtaagaagactagtgtttaaacctcgagcccggggagaagccctataaatgtcccgagtgcggaaagtccttctcacggcgcgatgaattgaacgtccatcagagaacacacaccggtaaaaaaactagtccgcggctcgagcccggggaaaaaccttataagtgtcccgagtgcggcaagagtttcagtcacaaaaacgcacttcagaatcatcagaggacacataccggtaagaaaactagtgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 67 - DNA, 57 bp, Artificial Sequence
  Synthetic construct
tcgacaggcccaggcggccctcgaggatatcatgatgactagtggccaggccggccc

SEQ ID NO: 68 - DNA, 57 bp, Artificial Sequence
  Synthetic construct
aattgggccggcctggccactagtcatcatgatatcctcgagggccgcctgggcctg

SEQ ID NO: 69 - DNA, 6699 bp, Artificial Sequence
  Synthetic construct
gcttgcatgcaacttcttttctttttttttcttttctctctcccccgttgttgtctcaccatatccgcaatgacaaaaaaaatgatggaagacactaaaggaaaaaattaacgacaaagacagcaccaacagatgtcgttgttccagagctgatgaggggtatcttcgaacacacgaaactttttccttccttcattcacgcacactactctctaatgagcaacggtatacggccttccttccagttacttgaatttgaaataaaaaaagtttgccgctttgctatcaagtataaatagacctgcaattattaatcttttgtttcctcgtcattgttctcgttccctttcttccttgtttctttttctgcacaatatttcaagctataccaagcatacaatcaactccaagctttgcaaagatggataaagcggaattaattcccgagcctccaaaaaagaagagaaaggtcgaattgggtaccgccgccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagatctctcgacaggcccaggcggccctcgaggatatcatgatgactagtggccaggccggcccaattccagatctatgaatcgtagatactgaaaaaccccgcaagttcacttcaactgtgcatcgtgcaccatctcaatttctttcatttatacatcgttttgccttcttttatgtaactatactcctctaagtttcaatcttggccatgtaacctctgatctatagaattttttaaatgactagaattaatgcccatcttttttttggacctaaattcttcatgaaaatatattacgagggcttattcagaagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccggtcgaaattcccctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtaggaaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactacctgtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttggggatctcgactctagctagaggatcaattcgtaatcatggtcatagctgtttcctgtgtgaaattgttatccgctcacaattccacacaacatacgagccggaagcataaagtgtaaagcctggggtgcctaatgagtgaggtaactcacattaattgcgttgcgctcactgcccgctttccagtcgggaaacctgtcgtgccagctggattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgtt

SEQ ID NO: 70 - DNA, 6481 bp, Artificial Sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccgcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaagtccaaagcttctgaataagccctcgtaatatattttcatgaagaatttaggtccaaaaaaaagatgggcattaattctagtcatttaaaaaattctatagatcagaggttacatggccaagattgaaacttagaggagtatagttacataaaagaaggcaaaacgatgtataaatgaaagaaattgagatggtgcacgatgcacagttgaagtgaacttgcggggtttttcagtatctacgattcatagatctggaattgggccggcctggccactagtcatcatgatatcctcgagggccgcctgggcctgtcgagagatctctttttttgggtttggtggggtatcttcatcatcgaatagatagttatatacatcatccattgtagtggtattaaacatccctgtagtgattccaaacgcgttatacgcagtttggtccgtccaaccaggtgacagtggttttgaattattaccatcatcaattttactagccgtgatttcattattcatgaagttatcatgaacgttagaggaggcaattggttgtgaaagcgcttgagaatttgtttgagttgttatgaggttcggaccgttgctactgttagtgaaagtgaaggacaatgagctatcagcaatattcccactttgattaaaattggcggcggtacccaattcgacctttctcttcttttttggaggctcgggaattaattccgctttatccatctttgcaaagcttggagttgattgtatgcttggtatagcttgaaatattgtgcagaaaaagaaacaaggaagaaagggaacgagaacaatgacgaggaaacaaaagattaataattgcaggtctatttatacttgatagcaaagcggcaaactttttttatttcaaattcaagtaactggaaggaaggccgtataccgttgctcattagagagtagtgtgcgtgaatgaaggaaggaaaaagtttcgtgtgttcgaagatacccctcatcagctctggaacaacgacatctgttggtgctgtctttgtcgttaattttttcctttagtgtcttccatcattttttttgtcattgcggatatggtgagacaacaacgggggagagagaaaagaaaaaaaaagaaaagaagttgcatgcattcatgcgggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 71 - DNA, 6018 bp, Artificial Sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgagggtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctccaccgcggtggcggccgctctagaactagtggatcccccgggctgcaggaattcgatatcaagcttatcgataccgtcgacctcgagggggggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 72 - DNA, 23 bp, Artificial Sequence
  Synthetic construct
cgccgcatgcattcatgcaggcc

SEQ ID NO: 73 - DNA, 17 bp, Artificial Sequence
  Synthetic construct
tgcatgaatgcatgcgg

SEQ ID NO: 74 - DNA, 5021 bp, Artificial Sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccgcatgcattcatgcaggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 75 - DNA, 6408 bp, Artificial Sequence
  synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccgcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaagtccaaagcttctgaataagccctcgtaatatattttcatgaagaatttaggtccaaaaaaaagatgggcattaattctagtcatttaaaaaattctatagatcagaggttacatggccaagattgaaacttagaggagtatagttacataaaagaaggcaaaacgatgtataaatgaaagaaattgagatggtgcacgatgcacagttgaagtgaacttgcggggtttttcagtatctacgattcatagatctggaattgggccggcctggccactagtcatcatgatatcctcgagggccgcctgggcctgtcgagagatctctttttttgggtttggtggggtatcttcatcatcgaatagatagttatatacatcatccattgtagtggtattaaacatccctgtagtgattccaaacgcgttatacgcagtttggtccgtccaaccaggtgacagtggttttgaattattaccatcatcaattttactagccgtgatttcattattcatgaagttatcatgaacgttagaggaggcaattggttgtgaaagcgcttgagaatttgtttgagttgttatgaggttcggaccgttgctactgttagtgaaagtgaaggacaatgagctatcagcaatattcccactttgattaaaattggcggcggtacccaattcgacctttctcttcttttttggaggctcgggaattaattccgctttatccatctttgcagcggccgcttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 76 - DNA, 6308 bp, Artificial Sequence
  synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccgcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaagtccaaagcttctgaataagccctcgtaatatattttcatgaagaatttaggtccaaaaaaaagatgggcattaattctagtcatttaaaaaattctatagatcagaggttacatggccaagattgaaacttagaggagtatagttacataaaagaaggcaaaacgatgtataaatgaaagaaattgagatggtgcacgatgcacagttgaagtgaacttgcggggtttttcagtatctacgattcatagatctggaattgggccggcctggccactagtcatcatgatatcctcgagggccgcctgggcctgtcgagagatctctttttttgggtttggtggggtatcttcatcatcgaatagatagttatatacatcatccattgtagtggtattaaacatccctgtagtgattccaaacgcgttatacgcagtttggtccgtccaaccaggtgacagtggttttgaattattaccatcatcaattttactagccgtgatttcattattcatgaagttatcatgaacgttagaggaggcaattggttgtgaaagcgcttgagaatttgtttgagttgttatgaggttcggaccgttgctactgttagtgaaagtgaaggacaatgagctatcagcaatattcccactttgattaaaattggcggcggtacccaattcgacctttctcttcttttttggaggctcgggaattaattccgctttatccatctttgcagcggccgcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 77 - DNA, 7730 bp, Artificial Sequence
  synthetic construct
tctctggctaactagagaacccactgcttactggcttatcgaaattttaattaacgttggcaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggctaggtttaaacactagaaataattcttactgtcatgccaagtaagatgcttttctgtgctgcaatagcaggcatgctggggatgcggtgggctctatggcttctgaggcggaaagaactagacccagctttcttgtacaaagttggcattataagaaagcattgcttatcaatttgttgcaacgaacaggtcactatcagtcaaaataaaatcattatttgccatccaggtcgagtgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaaagatccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcggtacctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgctgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggccccgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggngaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgctagcatggatctcggggacgtctaactactaagcgagagtagggaactgccaggcatcaaataaaacgaaaggctcagtcggaagactgggcctttcgttttatctgttgtttgtcggtgaacgctctcctgagtaggacaaatccgccgggagcggatttgaacgttgtgaagcaacggcccggagggtggcgggcaggacgcccgccataaactgccaggcatcaaactaagcagaaggccatcctgacggatggcctttttgcgtttctacaaactcttcctgttagttagttacttaagctcgggccccaaataatgattttattttgactgatagtgacctgttcgttgcaacaaattgataagcaatgcttttttataatgccaactttgtacaaaaaagcaggcttcgaaggagatagaaccagatcttggaattctgcagatatcgaaatttgggaggggagccatcaaagaagcctgggagcagcagttccagggaaaaaggagaatgtgatggccagagagccaaaagaaaaagtagttgaaggagtgctcagcactaggcatctgaactgaatgctgtggcaggctcactggccacaaacaatagggagctggtggaggccttgacgaggaccatttcaacaaactggtgggcttaaaatccggaagaaacagttgaacaaatcattttgacgccttttataaaccacacaagcttattccaaacccgttactggcctaactgatttaagtccctttcccatctgatcctcagagattctaagggacttagcctatccatgactcttcgtcctgcttctcacctcccatgattgccctaacgatgtgaaagtgctttcaaacaaagatgcccaagaaagaaggtaggcaaatgtgcaagcattagtttgtagtacgctattactgtatttcaccttgcactctctagtttccttcgtgctccctcaatatccaactcttaataaattcatggctcccggtgagcattcatcaattctcattccacgcctttagcccttcccgttcccgcccaactctcgctccctcccctggccaaatctctaacctgcaaggctaattccgaattccaaatcggaagcaagagggcggggccccgtgagaggcgatggattgctccagtccgttcccgacgcactgtgcgcatgcgctggtcctccgcggaccgttcgtgctgcccgcctagaaagggtgaagtggttgtttccgtgacggactgagtacgggtgcctgtcaggctcttgcggaagtccatgcgccattgggagggcctcggccgcggctctgtgcccttgctgctgagggccacttcctgggtcattcctggaccgggagccgggctggggctcacacgggggctcccgcgtggccgtctcggcgcctgcgtgacctccccgccggcgggctcgagcccaagcttggtaccgagctcggatccagccaccatgggagtcaaagttctgtttgccctgatctgcatcgctgnggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagctggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccctggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacaaccggtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagc

SEQ ID NO: 78 - DNA, 6083 bp, Artificial Sequence
  Synthetic construct
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagagatatcatggatgctaagtccctgacagcgtggagccgcacactggttaccttcaaagatgttttcgtggatttcacccgcgaagagtggaaactgctggataccgcacagcagattgtgtatcgcaacgttatgctggaaaactacaagaatctggttagcctgggctatcagctgacaaaacccgacgtcatcctgcgtctggaaaagggtgaagagccgtggctggttgaacgggagattcaccaggagacacatcctgattctgaaactgcctttgagatcaaaagctccgtcagtccgaaaaagaaacgtaaagtggggctcgagcccggggaaaagccatataaatgccccgagtgcggcaaatcattcagccaaagtagcaacttagtaagacaccagcgcacccataccggggaaaagccatataaatgccccgagtgcggcaaatcattcagccaaagtagcaacttagtaagacaccagcgcacccataccggggaaaagccatataaatgccccgagtgcggcaaatcattcagccaaagtagcaacttagtaagacaccagcgcacccataccggtgagcagaaactcatctctgaagaagatctggaacaaaagttgatttcagaagaagatctggaacagaagctcatctctgaggaagatctgtaagcggccgcgaattccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 79 - DNA, 5916 bp, Artificial Sequence
  Synthetic construct
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagagatatcatgccgaaaaagaaacgtaaagtggggctcgagcccggggaaaagccatataaatgccccgagtgcggcaaatcattcagccaaagtagcaacttagtaagacaccagcgcacccataccggggaaaagccatataaatgccccgagtgcggcaaatcattcagccaaagtagcaacttagtaagacaccagcgcacccataccggggaaaagccatataaatgccccgagtgcggcaaatcattcagccaaagtagcaacttagtaagacaccagcgcacccataccggtggcggcagcggcggcagcgaattccgcacactggttaccttcaaagatgttttcgtggatttcacccgcgaagagtggaaactgctggataccgcacagcagattgtgtatcgcaacgttatgctggaaaactacaagaatctggttagcctgggctatggatccgagcagaaactcatctctgaagaagatctggaacaaaagttgatttcagaagaagatctggaacagaagctcatctctgaggaagatctgtaagcggccgcaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 80 - DNA, 5897 bp, Artificial Sequence
  Synthetic construct
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagagatatcatggcggcggcggttcggatgaacatccagatgctgctggaggcggccgactatctggagcggcgggagagagaagctgaacatggttatgcctccatgttaccatacccgaaaaagaaacgtaaagtggggctcgagcccggggaaaagccatataaatgccccgagtgcggcaaatcattcagccaaagtagcaacttagtaagacaccagcgcacccataccggggaaaagccatataaatgccccgagtgcggcaaatcattcagccaaagtagcaacttagtaagacaccagcgcacccataccggggaaaagccatataaatgccccgagtgcggcaaatcattcagccaaagtagcaacttagtaagacaccagcgcacccataccggtgagcagaaactcatctctgaagaagatctggaacaaaagttgatttcagaagaagatctggaacagaagctcatctctgaggaagatctgtaagcggccgcgaattccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 81 - DNA, 6198 bp, Artificial Sequence
  Synthetic construct
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagagatatcatgccgaaaaagaaacgtaaagtggggctcgagcccggggaaaagccctacaagtgccctgagtgtgggaagtccttttcttcaagacgcacgtgccgcgctcaccagcggacacataccggggagaagccctataaatgtccagaatgtggaaagtcctttagcacgtcagggaacttagtaagacaccagcgaactcataccggggagaagccatataaatgtcccgagtgtggcaagtccttttctagatcagataatttagtaagacatcagagaacgcacaccggggaaaagccctacaagtgcccggaatgcggcaagtcttttagcaccagcggacatttagtaagacaccagagaacccacaccggggaaaaaccctataaatgccccgagtgtggtaagtcattctctcaaagcggggatttaagaagacaccagagaacccacaccggggaaaaaccgtataaatgtcctgagtgcggtaagtctttttccgactgtagagacttagcgagacaccaacgtactcataccggtggcggcagcggcggcagcgaattcgggcgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaattaacggatccgagcagaaactcatctctgaagaagatctggaacaaaagttgatttcagaagaagatctggaacagaagctcatctctgaggaagatctgtaagcggccgcaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 82 - DNA, 5185 bp, Artificial Sequence
  Synthetic construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcaccaccaccaccaccacggctatggccgcaaaaaacgccgccagcgccgccgcggctatccgtatgatgtgccggattatgccccatgggatatcggggatccgaattctgtacaggccttggcgcgcctgcaggcgagctccgtcgacaagcttgcggccgcactcgagcaccaccaccaccaccaccaccactaattgattaatacctaggctgctaaacaaagcccgaaaggaagctgagttggctgctgccaccgctgagcaataactagcataaccccttggggcctctaaacgggtcttgaggggttttttgctgaaaggaggaactatatccggat

SEQ ID NO: 83 - DNA, 5956 bp, Artificial Sequence
  synthetic construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcaccaccaccaccaccacggctatggccgcaaaaaacgccgccagcgccgccgcggctatccgtatgatgtgccggattatgccccatgggatatcatgccgaaaaagaaacgtaaagtggggctcgagcccggggngaagccctataaatgccctgaatgcgggaaatctttctcttctaagaaggcactcacagaacaccagcggacacacaccggggaaaaaccgtacaagtgtcctgagtgcgggaagagtttctccgatccgggccacttagtaagacatcagaggacacataccggggagaagccatataaatgtcccgagtgtggcaagtccttttctagatcagataatttagtaagacatcagagaacgcacaccggggagaagccatataaatgtcccgagtgtggcaagtccttttctagatcagataatttagtaagacatcagagaacgcacaccggggaaaagccatataaatgccccgagtgcggcaaatcattcagccaaagtagcaacttagtaagacaccagcgcacccataccggggaaaaaccgtacaagtgtcctgagtgcgggaagagtttctccgatccgggccacttagtaagacatcagaggacacataccggtggcggcagcggcggcagcgaattcgggcgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaattaacggatccgagcagaaactcatctctgaagaagatctggaacaaaagttgatttcagaagaagatctggaacagaagctcatctctgaggaagatctgtaagcggccgcactcgagcaccaccaccaccaccaccaccactaattgattaatacctaggctgctaaacaaagcccgaaaggaagctgagttggctgctgccaccgctgagcaataactagcataaccccttggggcctctaaacgggtcttgaggggttttttgctgaaaggaggaactatatccggat

SEQ ID NO: 84 - DNA, 5956 bp, Artificial Sequence
  synthetic construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcaccaccaccaccaccacggctatggccgcaaaaaacgccgccagcgccgccgcggctatccgtatgatgtgccggattatgccccatgggatatcatgccgaaaaagaaacgtaaagtggggctcgagcccggggagaaaccatacaaatgccccgagtgtggaaagtcatttagtgatccaggcgcattagtaagacatcagcggacacataccggggagaagccatataaatgtcccgagtgtggcaagtccttttctagatcagataatttagtaagacatcagagaacgcacaccggggagaagccctacaagtgtccagaatgcggaaagagtttctccagaagtgacaaattagtaagacaccagagaacccataccggggaaaaaccgtacaagtgtcctgagtgcgggaagagtttctccgatccgggccacttagtaagacatcagaggacacataccggggaaaaaccgtataaatgtcctgagtgcggtaagtctttttccgactgtagagacttagcgagacaccaacgtactcataccggggagaaaccatacaaatgtcccgaatgtggcaagagtttcagcagtaaaaagcatctcgctgagcatcagagaactcacaccggtggcggcagcggcggcagcgaattcgggcgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaattaacggatccgagcagaaactcatctctgaagaagatctggaacaaaagttgatttcagaagaagatctggaacagaagctcatctctgaggaagatctgtaagcggccgcactcgagcaccaccaccaccaccaccaccactaattgattaatacctaggctgctaaacaaagcccgaaaggaagctgagttggctgctgccaccgctgagcaataactagcataaccccttggggcctctaaacgggtcttgaggggttttttgctgaaaggaggaactatatccggat

SEQ ID NO: 85 - DNA, 5956 bp, Artificial Sequence
  synthetic construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcaccaccaccaccaccacggctatggccgcaaaaaacgccgccagcgccgccgcggctatccgtatgatgtgccggattatgccccatgggatatcatgccgaaaaagaaacgtaaagtggggctcgagcccggggagaagccgtacaagtgccctgaatgtggtaagtcattttcgagaagtgatgaattagtaagacaccagcggactcataccggggagaagccgtacaagtgccctgaatgtggtaagtcattttcgagaagtgatgaattagtaagacaccagcggactcataccggggagaagccctataaatgtccagaatgtggaaagtcctttagcacgtcagggaacttagtaagacaccagcgaactcataccggggaaaagccttacaaatgccccgaatgtgggaagagtttcagccggtctgataagctgaccgaacaccagagaactcataccggggagaagccctataaatgccctgaatgtggcaagagcttcagtactagcgggaatctcactgaacatcagcgaactcataccggggaaaaaccttacaagtgccctgagtgcggcaagagcttctctcaatcaagttcattagtaagacaccagaggactcataccggtggcggcagcggcggcagcgaattcgggcgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaattaacggatccgagcagaaactcatctctgaagaagatctggaacaaaagttgatttcagaagaagatctggaacagaagctcatctctgaggaagatctgtaagcggccgcactcgagcaccaccaccaccaccaccaccactaattgattaatacctaggctgctaaacaaagcccgaaaggaagctgagttggctgctgccaccgctgagcaataactagcataaccccttggggcctctaaacgggtcttgaggggttttttgctgaaaggaggaactatatccggat

SEQ ID NO: 86 - DNA, 5956 bp, Artificial Sequence
  synthetic construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcaccaccaccaccaccacggctatggccgcaaaaaacgccgccagcgccgccgcggctatccgtatgatgtgccggattatgccccatgggatatcatgccgaaaaagaaacgtaaagtggggctcgagcccggggagaaaccttataaatgcccagaatgcgggaaatcgttcagtcaaagagcacatttagaaagacatcaacggacccacaccggggaaaagccatataaatgccccgagtgcggcaaatcattcagccaaagtagcaacttagtaagacaccagcgcacccataccggggaaaagccctacaagtgtcctgagtgcggaaagtctttctccactagcggttcattagtaagacaccagaggacacacaccggggaaaaaccttacaagtgccctgagtgcggcaagagcttctctcaatcaagttcattagtaagacaccagaggactcataccggggagaagccatacaaatgccctgagtgtggaaagtcatttagccagcgagctaatctgcgggcccaccagcggacccacaccggggaaaagccatataaatgccccgagtgcggcaaatcattcagccaaagtagcaacttagtaagacaccagcgcacccataccggtggcggcagcggcggcagcgaattcgggcgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaattaacggatccgagcagaaactcatctctgaagaagatctggaacaaaagttgatttcagaagaagatctggaacagaagctcatctctgaggaagatctgtaagcggccgcactcgagcaccaccaccaccaccaccaccactaattgattaatacctaggctgctaaacaaagcccgaaaggaagctgagttggctgctgccaccgctgagcaataactagcataaccccttggggcctctaaacgggtcttgaggggttttttgctgaaaggaggaactatatccggat

SEQ ID NO: 87 - DNA, 5956 bp, Artificial Sequence
  synthetic construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcaccaccaccaccaccacggctatggccgcaaaaaacgccgccagcgccgccgcggctatccgtatgatgtgccggattatgccccatgggatatcatgccgaaaaagaaacgtaaagtggggctcgagcccggggagaaaccatacaaatgtcccgaatgtggcaagagtttcagcagtaaaaagcatctcgctgagcatcagagaactcacaccggggaaaaaccttacaagtgccctgagtgcggcaagagcttctctcaatcaagttcattagtaagacaccagaggactcataccggggaaaaaccgtacaagtgtcctgagtgcgggaagagtttctccgatccgggccacttagtaagacatcagaggacacataccggggagaaaccttataaatgcccagaatgcgggaaatcgttcagtcaaagagcacatttagaaagacatcaacggacccacaccggggaaaagccctacaagtgtcctgagtgcggaaagtctttctccactagcggttcattagtaagacaccagaggacacacaccggggaaaaaccttacaagtgccctgagtgcggcaagagcttctctcaatcaagttcattagtaagacaccagaggactcataccggtggcggcagcggcggcagcgaattcgggcgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaattaacggatccgagcagaaactcatctctgaagaagatctggaacaaaagttgatttcagaagaagatctggaacagaagctcatctctgaggaagatctgtaagcggccgcactcgagcaccaccaccaccaccaccaccactaattgattaatacctaggctgctaaacaaagcccgaaaggaagctgagttggctgctgccaccgctgagcaataactagcataaccccttggggcctctaaacgggtcttgaggggttttttgctgaaaggaggaactatatccggat

SEQ ID NO: 88 - DNA, 5956 bp, Artificial Sequence
  synthetic construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcaccaccaccaccaccacggctatggccgcaaaaaacgccgccagcgccgccgcggctatccgtatgatgtgccggattatgccccatgggatatcatgccgaaaaagaaacgtaaagtggggctcgagcccggggaaaagccctacaaatgccccgaatgtggtaagtctttttctaggaacgacaccttgacagaacaccagcggacccacaccggggaaaagccctacaagtgtcctgagtgcggaaagtctttctccactagcggttcattagtaagacaccagaggacacacaccggggaaaaaccgtacaagtgtcctgagtgcgggaagagtttctccgatccgggccacttagtaagacatcagaggacacataccggggagaaaccttataaatgcccagaatgcgggaaatcgttcagtcaaagagcacatttagaaagacatcaacggacccacaccggggaaaagccctacaagtgtcctgagtgcggaaagtctttctccactagcggttcattagtaagacaccagaggacacacaccggggaaaaaccttacaagtgccctgagtgcggcaagagcttctctcaatcaagttcattagtaagacaccagaggactcataccggtggcggcagcggcggcagcgaattcgggcgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaattaacggatccgagcagaaactcatctctgaagaagatctggaacaaaagttgatttcagaagaagatctggaacagaagctcatctctgaggaagatctgtaagcggccgcactcgagcaccaccaccaccaccaccaccactaattgattaatacctaggctgctaaacaaagcccgaaaggaagctgagttggctgctgccaccgctgagcaataactagcataaccccttggggcctctaaacgggtcttgaggggttttttgctgaaaggaggaactatatccggat

SEQ ID NO: 89 - DNA, 5956 bp, Artificial Sequence
  synthetic construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcaccaccaccaccaccacggctatggccgcaaaaaacgccgccagcgccgccgcggctatccgtatgatgtgccggattatgccccatgggatatcatgccgaaaaagaaacgtaaagtggggctcgagcccggggagaaaccatacaaatgtcccgaatgtggcaagagtttcagcagtaaaaagcatctcgctgagcatcagagaactcacaccggggaaaagccctacaagtgtcctgagtgcggaaagtctttctccactagcggttcattagtaagacaccagaggacacacaccggggagaaaccttataaatgcccagaatgcgggaaatcgttcagtcaaagagcacatttagaaagacatcaacggacccacaccggggaaaagccatataaatgccccgagtgcggcaaatcattcagccaaagtagcaacttagtaagacaccagcgcacccataccggggagaaaccatacaaatgccccgagtgtggaaagtcatttagtgatccaggcgcattagtaagacatcagcggacacataccggggaaaagccctacaagtgtcctgagtgcggaaagtctttctccactagcggttcattagtaagacaccagaggacacacaccggtggcggcagcggcggcagcgaattcgggcgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaattaacggatccgagcagaaactcatctctgaagaagatctggaacaaaagttgatttcagaagaagatctggaacagaagctcatctctgaggaagatctgtaagcggccgcactcgagcaccaccaccaccaccaccaccactaattgattaatacctaggctgctaaacaaagcccgaaaggaagctgagttggctgctgccaccgctgagcaataactagcataaccccttggggcctctaaacgggtcttgaggggttttttgctgaaaggaggaactatatccggat

(19) United States
(12) Patent Application Publication
(10) Pub. No.: US 2016/0039893 A1
(43) Pub. Date: Feb. 11, 2016

(54) ARTIFICIAL TRANSCRIPTION FACTORS FOR THE TREATMENT OF DISEASES CAUSED BY OPA1 HAPLOINSUFFICIENCY

(71) Applicant: ALIOPHTHA AG, Basel (CH)

(72) Inventors: Albert NEUTZNER, Schliengen (DE); Josef FLAMMER, Binningen (CH); Alice HUXLEY, Binningen (CH)

(73) Assignee: ALIOPHTHA AG, Basel (CH)

(21) Appl. No.: 14/781,710

(22) PCT Filed: Apr. 2, 2014
(86) PCT No.: PCT/EP2014/056590

§ 371 Date: Oct. 1, 2015

(30) Foreign Application Priority Data
Apr. 3, 2013 (EP) 13162189.8

(51) Int. Cl. C07K 14/435; A61K 38/17; A61K 47/48

(52) U.S. Cl. CPC C07K 14/435; A61K 47/48215; A61K 38/17; C07K 2319/09; C07K 2319/71; C07K 2319/81

(57) ABSTRACT

The invention relates to an artificial transcription factor comprising a polydactyl zinc finger protein targeting specifically the OPA1 promoter fused to an activatory protein domain, and a nuclear localization sequence. Artificial transcription factors directed against the OPA1 promoter are useful for the treatment of diseases associated with OPA1 haploinsufficiency, such as autosomal dominant optic atrophy, syndromic autosomal dominant optic atrophy plus and normal tension glaucoma.

### FIELD OF THE INVENTION

The invention relates to artificial transcription factors comprising a polydactyl zinc finger protein targeting specifically the OPA1 gene promoter fused to an activatory domain and a nuclear localization sequence, and their use in treating diseases such as autosomal dominant optic atrophy (ADOA) or syndromic ADOA plus, caused by mutations in OPA1 leading to haploinsufficiency.

### BACKGROUND OF THE INVENTION

Artificial transcription factors (ATFs) are proposed to be useful tools for modulating gene expression (Sera T., 2009, Adv Drug Deliv Rev 61, 513-526). Many naturally occurring transcription factors, influencing gene expression either through repression or activation of gene transcription, possess complex specific domains for the recognition of a certain DNA sequence. This makes them unattractive targets for manipulation if one intends to modify their specificity and target gene(s). However, a certain class of transcription factors contains several so called zinc finger (ZF) domains, which are modular and therefore lend themselves to genetic engineering. Zinc fingers are short (30 amino acids) DNA binding motifs targeting almost independently three DNA base pairs. A protein containing several such zinc fingers fused together is thus able to recognize longer DNA sequences. A hexameric zinc finger protein (ZFP) recognizes an 18 base pairs (bp) DNA target, which is almost unique in the entire human genome. Initially thought to be completely context independent, more in-depth analyses revealed some context specificity for zinc fingers (Klug A., 2010, Annu Rev Biochem 79, 213-231). Mutating certain amino acids in the zinc finger recognition surface altering the binding specificity of ZF modules resulted in defined ZF building blocks for most of 5′-GNN-3′, 5′-CNN-3′, 5′-ANN-3′, and some 5′-TNN-3′ codons (e.g. so-called Barbas modules, see Dreier B., Barbas C. F. 3^{rd} et al., 2005, J Biol Chem 280, 35588-35597). While early work on artificial transcription factors concentrated on a rational design based on combining preselected zinc fingers with a known 3 bp target sequence, the realization of a certain context specificity of zinc fingers necessitated the generation of large zinc finger libraries which are interrogated using sophisticated methods such as bacterial or yeast one hybrid, phage display, compartmentalized ribosome display or in vivo selection using FACS analysis.

Using such artificial zinc finger proteins, DNA loci within the human genome can be targeted with high specificity. Thus, these zinc finger proteins are ideal tools to transport protein domains with transcription-modulatory activity to specific promoter sequences resulting in the modulation of expression of a gene of interest. Suitable domains for the activation of gene transcription are herpes virus simplex VP16 (SEQ ID NO: 1) or VP64 (tetrameric repeat of VP16, SEQ ID NO: 2) domains (Beerli R. R. et al., 1998, Proc Natl Acad Sci USA 95, 14628-14633). Additional domains considered to confer transcriptional activation are CJ7 (SEQ ID NO: 3), p65-TA1 (SEQ ID NO: 4), SAD (SEQ ID NO: 5), NF-1 (SEQ ID NO: 6), AP-2 (SEQ ID NO: 7), SP1-A (SEQ ID NO: 8), SP1-B (SEQ ID NO: 9), Oct-1 (SEQ ID NO: 10), Oct-2 (SEQ ID NO: 11), Oct-2_{—}5× (SEQ ID NO: 12), MTF-1 (SEQ ID NO: 13), BTEB-2 (SEQ ID NO: 14) and LKLF (SEQ ID NO: 15). In addition, transcriptionally active domains of proteins defined by gene ontology GO:0001071 (http://amigo.geneontology.org/cgi-bin/amigo/term_details?term=GO:0001071) are considered to achieve transcriptional regulation of target proteins.

While small molecule drugs are not always able to selectively target a certain member of a given protein family due to the high conservation of specific features, biologicals offer great specificity as shown for antibody-based novel drugs. However, virtually all biologicals to date act extracellularly. Especially above mentioned artificial transcription factors would be suitable to influence gene transcription in a therapeutically useful way. However, the delivery of such factors to the site of action—the nucleus—is not easily achieved, thus hampering the usefulness of therapeutic artificial transcription factor approaches, e.g. by relaying on retroviral delivery with all the drawbacks of this method such as immunogenicity and the potential for cellular transformation (Lund C. V. et al., 2005, Mol Cell Biol 25, 9082-9091).

So called protein transduction domains (PTDs) were shown to promote protein translocation across the plasma membrane into the cytosol/nucleoplasm. Short peptides such as the HIV derived TAT peptide (SEQ ID NO: 16) and others were shown to induce a cell-type independent macropinocytotic uptake of cargo proteins (Wadia J. S. et al., 2004, Nat Med 10, 310-315). Upon arrival in the cytosol, such fusion proteins were shown to have biological activity. Interestingly, even misfolded proteins can become functional following protein transduction most likely through the action of intracellular chaperones.

Genetic mutations are at the heart of many inherited disorders. In general, such mutations can be classified into dominant or recessive regarding their mode of inheritance, with a dominant mutation being able to cause the disease phenotype even when only one gene copy—be it the maternal or the paternal—is affected, while for a recessive mutation to cause disease both, maternal and paternal, gene copies need to be mutated. Dominant mutations are able to cause disease by one of two general mechanisms, either by dominant-negative action or by haploinsufficiency. In case of a dominant-negative mutation, the gene product gains a new, abnormal function that is toxic and causes the disease phenotype. Examples are subunits of multimeric protein complexes that upon mutation prevent proper function of said protein complex. Diseases inherited in a dominant fashion can also be caused by haploinsufficiency, wherein the disease-causing mutation inactivates the affected gene, thus lowering the effective gene dose. Under these circumstances, the second, intact gene copy is unable to provide sufficient gene product for normal function. About 12,000 human genes are estimated to be haploinsufficient (Huang et al., 2010, PLoS Genet. 6(10), e1001154) with about 300 genes known to be associated with disease.

Neuronal survival critically depends on mitochondrial function with mitochondrial failure at the heart of many neurodegenerative disorders (Karbowski M., Neutzner A., 2012, Acta Neuropathol 123(2), 157-71). Besides their essential function in providing energy in the form of ATP, mitochondria are critically involved in calcium buffering, diverse catabolic as well as metabolic processes and also programmed cell death. This important function of mitochondria is mirrored in the many cellular mechanisms in place to maintain mitochondria and to prevent mitochondrial failure and subsequently cell death (Neutzner A. et al., 2012, Semin Cell Dev Biol 23, 499-508). A central role among these processes plays the maintenance of a dynamic mitochondrial network with a balanced mitochondrial morphology. This is achieved by the so called mitochondrial morphogens that promote either fission of mitochondria in the case of Drp1, Fis1, Mff, MiD49 and MiD51—or fusion of mitochondrial tubules in the case of Mfn1, Mfn2 and OPA1. Balancing mitochondrial morphology is essential since loss of mitochondrial fusion is known to promote the loss of ATP production and sensitizes cells to apoptotic stimuli connecting this process to neuronal cell death associated with neurodegenerative disorders.

A key player in the process of mitochondrial fusion is optic atrophy 1 or OPA1. OPA1 is a large GTPase encoded by the OPA1 gene and essential for mitochondrial fusion. In addition, OPA1 plays an important role in maintaining the internal, mitochondrial structure as component of the cristae. It was shown that downregulation of OPA1 gene expression causes mitochondrial fragmentation due to a loss of fusion and sensitizes cells to apoptotic stimuli. Mutations in OPA1 were identified to be responsible for about 70% of Kjer's optic neuropathy or autosomal dominant atrophy (ADOA). In most populations, ADOA is prevalent between 1/10,000 and 3/100,000 and is characterized by a slowly progressing decrease in vision starting in early childhood. The visual impairment ranges from mild to legally blind, is irreversible and is caused by the slow degeneration of the retinal ganglion cells (RGCs). In most cases, ADOA is non-syndromic, however, in about 15% of patients extra-ocular, neuro-muscular manifestations such as sensori-neural hearing loss are encountered. Until now, no viable treatment for this disease is available. Interestingly, certain OPA1 alleles were connected to normal tension, but not high tension glaucoma, highlighting again the importance of OPA1 for maintaining normal mitochondrial physiology.

### SUMMARY OF THE INVENTION

The invention relates to an artificial transcription factor comprising a polydactyl zinc finger protein targeting the OPA1 promoter fused to an activatory protein domain and a nuclear localization sequence, and to pharmaceutical compositions comprising such an artificial transcription factor.

Furthermore, the invention relates to an artificial transcription factor comprising a polydactyl zinc finger protein targeting the OPA1 promoter fused to an activatory protein domain, a nuclear localization sequence and a protein transduction domain, and to pharmaceutical compositions comprising such an artificial transcription factor.

The invention also relates to the use of such artificial transcription factors in enhancing the expression of the OPA1 gene and for improving the generation of OPA1 gene product.

Furthermore, the invention relates to the use of such artificial transcription factors in the treatment of diseases caused or modified by low OPA1 levels, in particular for use in the treatment of eye diseases such as ADOA and ADOA plus. Likewise the invention relates to a method of treating a disease influenced by low OPA1 levels comprising administering a therapeutically effective amount of an artificial transcription factor of the invention to a patient in need thereof.

### BRIEF DESCRIPTION OF THE FIGURES

FIG. 1: Therapeutic approach for alleviating haploinsufficiency using transducible artificial transcription factors

(A) A haploinsufficient mutation (HM) causes a reduction of gene product generation (GP) form gene (G) under control of promoter (P) compared to the wild type situation (WT).

(B) An artificial transcription factor containing a hexameric zinc finger (ZF) protein targeting specifically a promoter (P) region of a haploinsufficient gene (G) fused to an activatory domain (RD) as well as a nuclear localization sequence (NLS) is transported into cells by the action of a protein transduction domain (PTD) such as TAT or others. Upon binding to the promoter of the mutated (HM) and wild type gene (G), the generation of gene product from the wild type gene copy is increased to substitute for the loss of gene product from the mutated gene copy.

(C) An artificial transcription factor containing a hexameric zinc finger (ZF) targeting specifically a promoter (P) region of a haploinsufficient gene (G) fused to an activatory domain (RD) as well as a nuclear localization sequence (NLS) is expressed by a cell following viral transduction of a cDNA coding for such artificial transcription factor. Upon binding to the promoter of the mutated (HM) and wild type gene (G), the generation of gene product from the wild type gene copy is increased to substitute for the loss of gene product from the mutated gene copy.

FIG. 2: OPA1 promoter region

Shown is the 5′ untranslated region of the OPA1 containing the OPA1 promoter (SEQ ID NO: 17). Highlighted are binding sites for artificial transcription factors of the invention (underlined, overlapping sites from position 85 to 102 and 91 to 108, from position 834 to 853, and from position 983 to 1000), and position 846 for transcription start (bold).

FIG. 3: Luciferase reporter assay to assess activity of OPA1-specific artificial transcription factors

HeLa cells were co-transfected with expression plasmids for OPA1_akt1 to OPA1_akt5 (panel A, labeled A1 to A5) or OPA1_akt6 to OPA1_akt10 (panel B, labeled A6 to A10) and a reporter plasmid containing Gaussia luciferase under control of the human OPA1 promoter and secreted alkaline phosphatase under control of the CMV promoter. Transfection with an inactive (modified) OPA1_akt1 (panel A) or an inactive (modified) OPA1_akt6 (panel B), wherein all zinc-coordinating cysteine residues in the zinc finger protein are exchanged to serine residue, served as controls (labeled C). Luciferase and secreted alkaline phosphatase activities were measured 48 hours after co-transfection.

Luciferase activity was normalized to secreted alkaline phosphatase activity and expressed as percentage of control (relative luciferase activity—RLA). Shown is the average of three independent experiments with the error bars depicting SD.

### DETAILED DESCRIPTION OF THE INVENTION

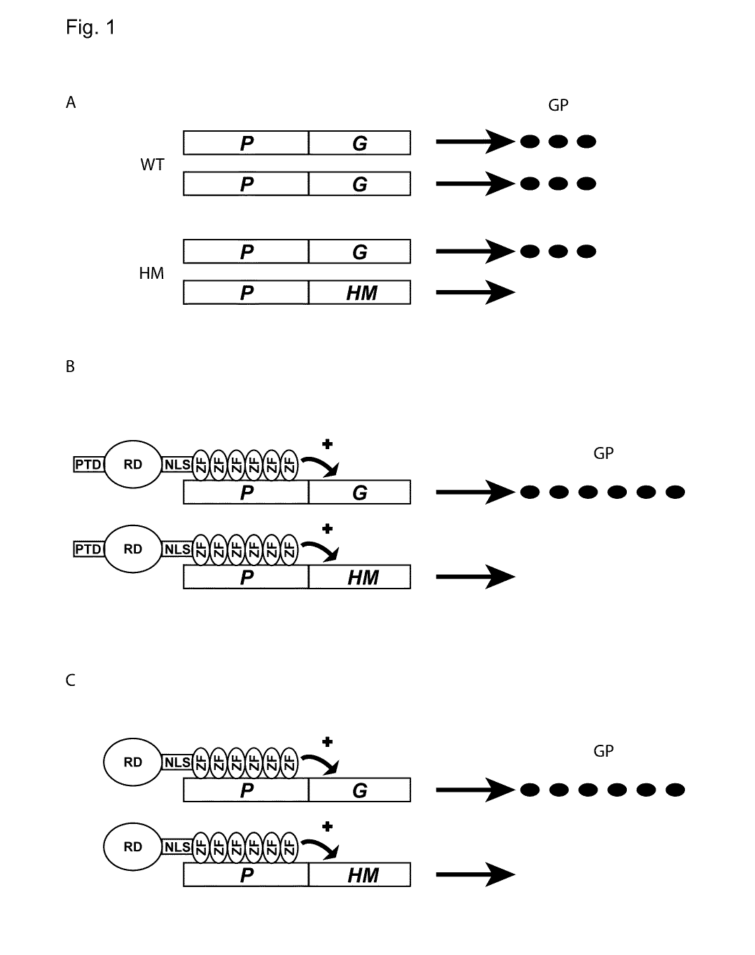

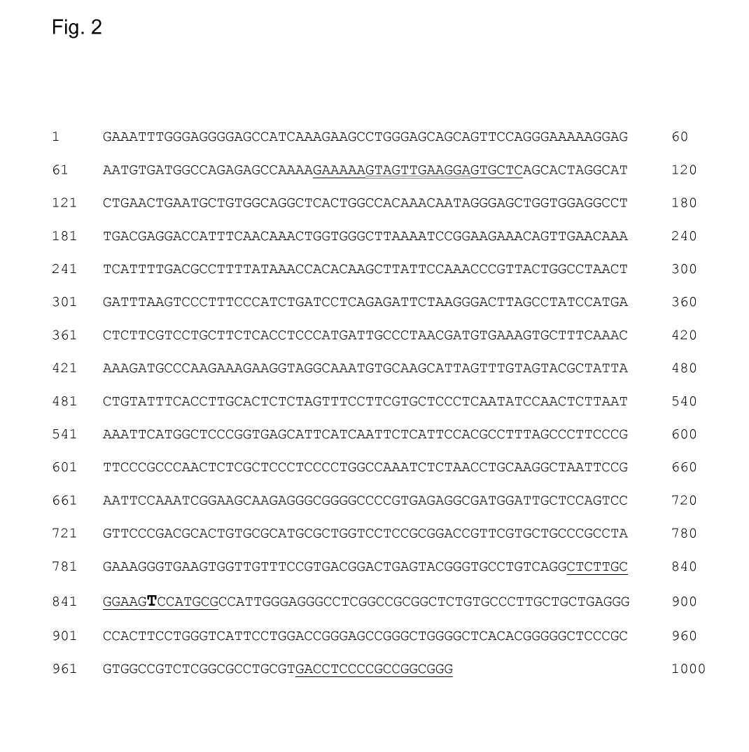

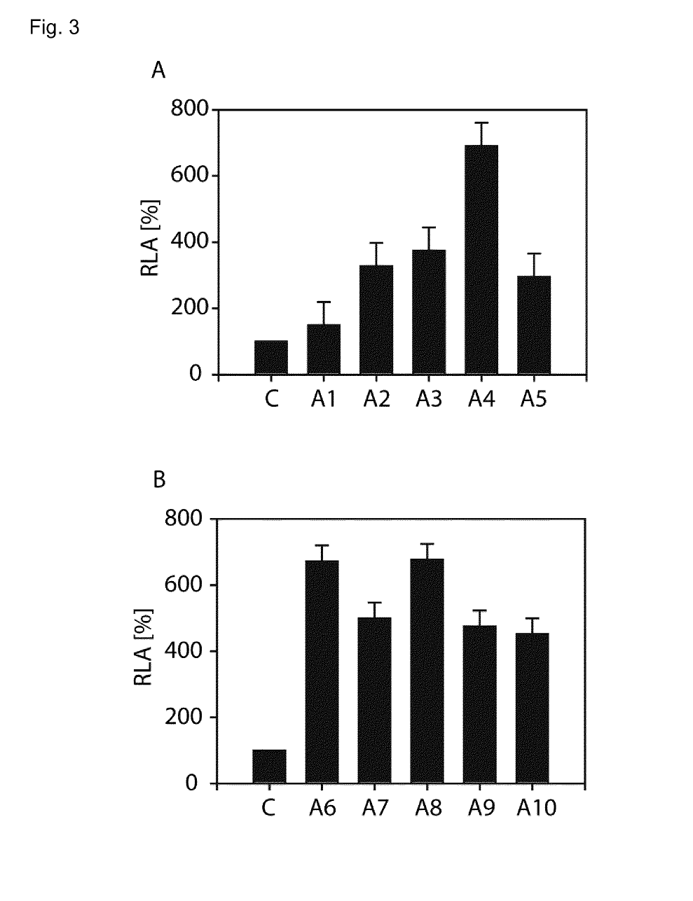

The invention relates to an artificial transcription factor (ATF) comprising a polydactyl zinc finger protein (ZFP) targeting specifically the OPA1 promoter (SEQ ID NO: 17) fused to an activatory protein domain, a nuclear localization sequence (NLS), and optionally a protein transduction domain (PTD), and to pharmaceutical compositions comprising such an artificial transcription factor (FIG. 1).

In the context of the present invention, a promoter is defined as the regulatory region of a gene. This definition corresponds to the general definition in the art. Also in the context of the present invention, a haploinsufficient promoter is defined as a promoter capable of causing the production of sufficient gene product in all cell types under all circumstances only if two functional gene copies are present in the genome. Thus, mutation of one gene copy of a haploinsufficient gene causes insufficient gene product generation in some or all cells of an organism under some or all physiological circumstances. In the context of the present invention, a gene is defined as genomic region containing regulatory sequences as well as sequences for the gene product resulting in the production of proteins or RNAs. This definition again corresponds to the general definition in the art.

Protein transduction domain-mediated, intracellular delivery of artificial transcription factors is a new way of taking advantage of the high selectivity of biologicals to target pathophysiological relevant molecules in a novel fashion. For diseases caused by haploinsufficiency of OPA1, such as ADOA or ADOA plus, no treatment using the current approaches, e.g. small molecule drugs, is conceivable, since insufficient gene expression is the root cause for such disorders. However, by pairing artificial transcription factor technology with advanced drug targeting in the form of protein transduction domains (PTD), haploinsufficiency of OPA1 can be addressed directly at the molecular level by transporting an activating artificial transcription factor and enhancing transcription of the remaining functional gene copy to levels that would be reached if both gene copies were functional.

Protein transduction domains considered are HIV TAT, the peptide mT02 (SEQ ID NO: 18), the peptide mT03 (SEQ ID NO: 19), the R9 peptide (SEQ ID NO: 20), the ANTP domain (SEQ ID NO: 21) or other peptides capable of transporting cargo across the plasma membrane.

Furthermore, modification of artificial transcription factors of the invention with polyethylene glycol is considered to reduce immunogenicity. In addition, application of artificial transcription factors of the invention to immune privileged organs such as the eye and the brain will avoid any immune reaction, and induce whole body tolerance to the artificial transcription factors. For the treatment of chronic diseases outside of immune privileged organs, induction of immune tolerance through prior intraocular injection is considered.

Dominant optic atrophy is caused by mutations in the OPA1 gene leading to haploinsufficiency. Dominant optic atrophy patients suffer from progressive vision loss ultimately causing blindness due to the progressive loss of retinal ganglion cells forming the optic nerve. Interestingly, most dominant optic atrophy patients do not present with extra-ocular symptoms. Only a small subset of patients suffer from a so-called dominant optic atrophy plus phenotype with additional extra-ocular neurological symptoms such as spastic paraplegia and hearing impairment. OPA1 is involved in maintaining mitochondrial function on a structural level by stabilizing the structure of the inner mitochondrial cristae and by promoting fusion between mitochondrial tubules. Since mitochondria are the main producer of cellular energy in form of ATP, OPA1 is necessary to maintain cellular energy levels. Loss of OPA1 function is known to promote cell death via apoptotic mechanisms. In almost all cells of the human body one functional copy of the OPA1 gene is sufficient to produce enough OPA1 protein to maintain mitochondrial function at a sufficient level. However, the particularly energy-hungry retinal ganglion cells have special needs regarding the state of their mitochondria and therefore depend on levels of OPA1 that cannot be produced by one OPA1 gene copy, hence, haploinsufficient OPA1 mutations are associated with retinal ganglion cell death and result in vision loss and blindness. Using artificial transcription factors of the invention, OPA1 protein levels can be increased in retinal ganglion cells by enhancing production of OPA1 protein from the remaining, functional OPA1 gene above normal levels, thus restoring mitochondrial function, preventing retinal ganglion cell death and associated vision loss.

Haploinsufficiency of OPA1 could in theory be treated by classical gene therapy approaches through supplying an additional, functional copy of the mutated OPA1 gene by means of viral transfer, thus increasing gene dosage. However, currently available viral vectors deemed safe for gene therapy are not capable of transporting gene larger than about 5 to 8 kilobases. While this is sufficient for some genes, the OPA1 gene is considerable larger than 8 kilobases and is therefore not a candidate for gene therapy employing currently available vectors. In addition, exact regulation of gene expression is not achievable using gene therapy with the potential of gross overexpression of the delivered gene and associated toxic side effects.

This limitation of viral transfer does not apply to artificial transcription factors of the present invention. The size of the haploinsufficient gene is not relevant for the therapeutic approach described in the present invention (FIG. 1) with even the largest genes amenable for modulation by artificial transcription factors. In addition, the extent to which gene expression is increased by artificial transcription factors of the invention is modulated through dosing the artificial transcription factor accordingly or by employing alternative activating domains with higher or lower activity in term of transcriptional modulation. In addition, the OPA1 mRNA is subject to extensive alternative splicing causing the production of several OPA1 isoforms which are all necessary for OPA1 to perform its function. Especially, differential proteolytic processing of various OPA1 isoforms is an essential mechanistic prerequisite for OPA1 to perform its function.

Using viral delivery of artificial transcription factors of the present invention for increasing OPA1 mRNA production in a functional gene copy will allow for this essential process to occur, thus providing a functional cure for diseases caused by OPA1 haploinsufficiency.

Classes of small molecules traditionally used as pool for therapeutic agents are not suitable for targeted modulation of gene expression. Thus, many promising drug targets and associated diseases are not amenable to classical pharmaceutical approaches. In contrast, artificial transcription factors of the invention all belong to the same substance class with a highly defined overall composition. Two hexameric zinc finger protein-based artificial transcription factors targeting two very diverse promoter sequences still have a minimal amino acid sequence identity of 85% with an overall similar tertiary structure and can be generated via a standardized method (as described below) in a fast and economical manner. Thus, artificial transcription factors of the invention combine, in one class of molecule, exceptionally high specificity for a very wide and diverse set of targets with overall similar composition. In addition, formulation of artificial transcription factors of the invention into drugs can rely on previous experience further expediting the drug development process.

The invention also relates the use of such artificial transcription factors in treating diseases caused by mutations in OPA1 leading to haploinsufficiency of OPA1, for which the polydactyl zinc finger protein is specifically targeting the OPA1 promoter region. Likewise the invention relates to a method of treating diseases comprising administering a therapeutically effective amount of an artificial transcription factor of the invention to a patient in need thereof, wherein the disease to be treated is caused by haploinsufficiency of the OPA1 gene, and for which the polydactyl zinc finger protein is specifically targeting the OPA1 promoter.

Polydactyl zinc finger proteins considered are tetrameric, pentameric, hexameric, heptameric or octameric zinc finger proteins. “Tetrameric”, “pentameric”, “hexameric”, “heptameric” and “octameric” means that the zinc finger protein consists of four, five, six, seven and eight partial protein structures, respectively, each of which has binding specificity for a particular nucleotide triplet. Preferably the artificial transcription factors comprise hexameric zinc finger proteins.

Selection of Target Sites within the OPA1 Promoter Region

Target site selection is crucial for the successful generation of a functional artificial transcription factor. For an artificial transcription factor to modulate OPA1 gene expression in vivo, it must bind its target site in the genomic context of the OPA1 gene. This necessitates the accessibility of the DNA target site, meaning chromosomal DNA in this region is not tightly packed around histones into nucleosomes and no DNA modifications such as methylation interfere with artificial transcription factor binding. While large parts of the human genome are tightly packed and transcriptionally inactive, the immediate vicinity of the transcriptional start site (−1000 to +200 bp) of an actively transcribed gene must be accessible for endogenous transcription factors and the transcription machinery such as RNA polymerases. Thus, selecting a target site in this area of any given target gene will allow the successful generation of an artificial transcription factor with the desired function in vivo.

Selection of Target Sites within the Human OPA1 Gene Promoter

A region 1000 bp upstream of the start codon of the human OPA1 open reading frame (FIG. 2) was analyzed for the presence of potential 18 bp target sites with the general composition of (G/C/ANN)₆, wherein G is the nucleotide guanine, C the nucleotide cytosine, A the nucleotide adenine and N stands for each of the four nucleotide guanine, cytosine, adenine and thymine. Four target sites, OPA_TS1 (SEQ ID NO: 22), OPA_TS2 (SEQ ID NO: 23), OPA_TS3 (SEQ ID NO: 24), and OPA_TS4 (SEQ ID NO: 25) were chosen.

### Transducible Artificial Transcription Factors Targeting the OPA1 Gene Promoter

Specific hexameric zinc finger proteins were composed of the so called Barbas zinc finger module set (Gonzalez B., 2010, Nat Protoc 5, 791-810) using the ZiFit software v3.3 (Sander JD., Nucleic Acids Research 35, 599-605) or were selected from zinc finger protein libraries using yeast one hybrid techniques. To generate activating transducible artificial transcription factors targeting the OPA1 gene promoter, hexameric zinc finger proteins ZFP_OPA1_{—}1 (SEQ ID NO: 26), ZFP_OPA1_{—}2 (SEQ ID NO: 27), ZFP_OPA1_{—}3 (SEQ ID NO: 28), ZFP_OPA1_{—}4 (SEQ ID NO: 29), ZFP_OPA1_{—}5 (SEQ ID NO: 30), ZFP_OPA1_{—}6 (SEQ ID NO: 31), ZFP_OPA1_{—}7 (SEQ ID NO: 32), ZFP_OPA1_{—}8 (SEQ ID NO: 33), ZFP_OPA1_{—}9 (SEQ ID NO: 34), ZFP_OPA1_{—}10 (SEQ ID NO: 35), ZFP_OPA1_{—}11 (SEQ ID NO: 36), ZFP_OPA1_{—}12 (SEQ ID NO: 37), ZFP_OPA1_{—}13 (SEQ ID NO: 38), ZFP_OPA1_{—}14 (SEQ ID NO: 39), ZFP_OPA1_{—}15 (SEQ ID NO: 40), ZFP_OPA1_{—}16 (SEQ ID NO: 41), ZFP_OPA1_{—}17 (SEQ ID NO: 42), and ZFP_OPA1_{—}18 (SEQ ID NO: 43), were fused to the transcription activating domain VP64 yielding artificial transcription factors OPA_akt1 (SEQ ID NO: 44), OPA_akt2 (SEQ ID NO: 45), OPA_akt3 (SEQ ID NO: 46), OPA_akt4 (SEQ ID NO: 47), OPA_akt5 (SEQ ID NO: 48), OPA_akt6 (SEQ ID NO: 49), OPA_akt7 (SEQ ID NO: 50), OPA_akt8 (SEQ ID NO: 51), OPA_akt9 (SEQ ID NO: 52), OPA_akt10 (SEQ ID NO: 53), OPA_akt11 (SEQ ID NO: 54), OPA_akt12 (SEQ ID NO: 55), OPA_akt13 (SEQ ID NO: 56), OPA_akt14 (SEQ ID NO: 57), OPA_akt15 (SEQ ID NO: 58), OPA_akt16 (SEQ ID NO: 59), OPA_akt17 (SEQ ID NO: 60), and OPA_akt18 (SEQ ID NO: 61) also containing a NLS and a 3×myc epitope tag.

Considered are also artificial transcription factors of the invention containing pentameric or hexameric, heptameric or octameric zinc finger proteins, wherein individual zinc finger modules are exchanged to improve binding affinity towards target sites of the OPA1 promoter gene or to alter the immunological profile of the zinc finger protein for improved tolerability.

The artificial transcription factors targeting the OPA1 promoter according to the invention also comprise a zinc finger protein based on the zinc finger module composition as disclosed in SEQ ID NO: 26 and 43, wherein individual amino acids are exchanged in order to minimize potential immunogenicity while retaining binding affinity to the intended target site.

The artificial transcription factors of the invention might also contain other protein domains capable of increasing gene transcription as defined by gene ontology GO:0001071, such as VP16, VP64 (tetrameric repeat of VP16), CJ7, p65-TA1, SAD, NF-1, AP-2, SP1-A, SP1-B, Oct-1, Oct-2, Oct-2_{—}5x, MTF-1, BTEB-2, LKLF. and others, preferably VP64 or AP-2.

Further, the artificial transcription factors of the invention comprise a nuclear localization sequence (NLS). Nuclear localization sequences considered are amino acid motifs conferring nuclear import through binding to proteins defined by gene ontology GO:0008139, for example clusters of basic amino acids containing a lysine residue (K) followed by a lysine (K) or arginine residue (R), followed by any amino acid (X), followed by a lysine or arginine residue (K-K/R-X-K/R consensus sequence, Chelsky D. et al., 1989 Mol Cell Biol 9, 2487-2492) or the SV40 NLS (SEQ ID NO: 62), with the SV40 NLS being preferred.

Artificial transcription factors directed to a promoter region of the OPA1 gene, but without the protein transduction domain, are also a subject of the invention. They are intermediates for the artificial transcription factors of the invention as defined hereinbefore, or may be used as such.

Considered are alternative delivery methods for artificial transcription factors of the invention in form of nucleic acids transferred by transfection or via viral vectors, such as herpes virus-, adeno virus- and adeno-associated virus-based vectors.

The domains of the artificial transcription factors of the invention may be connected by short flexible linkers. A short flexible linker has 2 to 8 amino acids, preferably glycine and serine. A particular linker considered is GGSGGS (SEQ ID NO: 63). Artificial transcription factors may further contain markers to ease their detection and processing.

Assessing OPA1 Upregulation and Improved Mitochondrial Activity Following Treatment with Artificial Transcription Factor Targeting the OPA1 Promoter

HeLa cells treated with OPA1 promoter specific artificial transcription factor will be compared with buffer control treated cells and protein levels of OPA1 will be assessed by quantitative infrared-fluorescence based Western blot using specific anti-OPA1 antibodies. Increases in OPA1 protein levels are indicative of increased production of OPA1 following treatment with artificial transcription factor. To measure beneficial effect of treatment with OPA1 specific artificial transcription factor, mitochondrial fidelity and cellular survival is being assessed. To this end, cells treated with OPA1 specific artificial transcription factor are compared to control treated cells in terms of mitochondrial reactive oxygen production following oxidative insult triggered through treatment with the mitochondrial poison rotenone. Mitochondrial reactive oxygen production is measured using flow cytometry and the reactive oxygen specific dye MitoSox. In addition, mitochondrial membrane potential as parameter of mitochondrial health is measured by flow cytometric detection of potential-sensitive TMRE fluorescence. A lowering of reactive oxygen species production or an increase in mitochondrial membrane potential in artificial transcription factor treated cells compared to control cells is indicative of a beneficial activity of the OPA1-targeting artificial transcription factor. Furthermore, sensitivity towards apoptotic induction by staurosporine, rotenone and actinomycin D of cells treated with either OPA1-targeting artificial transcription factor or control treated cells is measured. To this end, release of cytochrome c as indicator of apoptotic cell death is measured using fluorescence microscopy of treated cells and compared to control cells.

### Attachment of a Polyethylene Glycol Residue

The covalent attachment of a polyethylene glycol residue (PEGylation) to an artificial transcription factor of the invention is considered to increase solubility of the artificial transcription factor, to decrease its renal clearance, and control its immunogenicity. Considered are amine as well as thiol reactive polyethylene glycols ranging in size from 1 to 40 Kilodalton. Using thiol reactive polyethylene glycols, site-specific PEGylation of the artificial transcription factors is achieved. The only essential thiol group containing amino acids in the artificial transcription factors of the invention are the cysteine residues located in the zinc finger modules essential for zinc coordination. These thiol groups are not accessible for PEGylation due their zinc coordination, thus, inclusion of one or several cysteine residues into the artificial transcription factors of the invention provides free thiol groups for PEGylation using thiol-specific polyethylene glycol reagents.

### Pharmaceutical Compositions

The present invention relates also to pharmaceutical compositions comprising an artificial transcription factor as defined above. Pharmaceutical compositions considered are compositions for parenteral systemic administration, in particular intravenous administration, compositions for inhalation, and compositions for local administration, in particular ophthalmic-topical administration, e.g. as eye drops, or intravitreal, subconjunctival, parabulbar or retrobulbar administration, to warm-blooded animals, especially humans. Particularly preferred are eye drops and compositions for intravitreal, subconjunctival, parabulbar or retrobulbar administration. The compositions comprise the active ingredient alone or, preferably, together with a pharmaceutically acceptable carrier. Further considered are slow-release formulations. The dosage of the active ingredient depends upon the disease to be treated and upon the species, its age, weight, and individual condition, the individual pharmacokinetic data, and the mode of administration.

Further considered are pharmaceutical compositions useful for oral delivery, in particular compositions comprising suitably encapsulated active ingredient, or otherwise protected against degradation in the gut. For example, such pharmaceutical compositions may contain a membrane permeability enhancing agent, a protease enzyme inhibitor, and be enveloped by an enteric coating.

The pharmaceutical compositions comprise from approximately 1% to approximately 95% active ingredient. Unit dose forms are, for example, ampoules, vials, inhalers, eye drops and the like.

The pharmaceutical compositions of the present invention are prepared in a manner known per se, for example by means of conventional mixing, dissolving or lyophilizing processes.

Preference is given to the use of solutions of the active ingredient, and also suspensions or dispersions, especially isotonic aqueous solutions, dispersions or suspensions which, for example in the case of lyophilized compositions comprising the active ingredient alone or together with a carrier, for example mannitol, can be made up before use. The pharmaceutical compositions may be sterilized and/or may comprise excipients, for example preservatives, stabilizers, wetting agents and/or emulsifiers, solubilizers, salts for regulating osmotic pressure and/or buffers and are prepared in a manner known per se, for example by means of conventional dissolving and lyophilizing processes. The said solutions or suspensions may comprise viscosity-increasing agents, typically sodium carboxymethylcellulose, carboxymethylcellulose, dextran, polyvinylpyrrolidone, or gelatins, or also solubilizers, e.g. Tween 80™ (polyoxyethylene(20)sorbitan mono-oleate).

Suspensions in oil comprise as the oil component the vegetable, synthetic, or semi-synthetic oils customary for injection purposes. In respect of such, special mention may be made of liquid fatty acid esters that contain as the acid component a long-chained fatty acid having from 8 to 22, especially from 12 to 22, carbon atoms. The alcohol component of these fatty acid esters has a maximum of 6 carbon atoms and is a monovalent or polyvalent, for example a mono-, di- or trivalent, alcohol, especially glycol and glycerol. As mixtures of fatty acid esters, vegetable oils such as cottonseed oil, almond oil, olive oil, castor oil, sesame oil, soybean oil and groundnut oil are especially useful.

The manufacture of injectable preparations is usually carried out under sterile conditions, as is the filling, for example, into ampoules or vials, and the sealing of the containers.

For parenteral administration, aqueous solutions of the active ingredient in water-soluble form, for example of a water-soluble salt, or aqueous injection suspensions that contain viscosity-increasing substances, for example sodium carboxymethylcellulose, sorbitol and/or dextran, and, if desired, stabilizers, are especially suitable. The active ingredient, optionally together with excipients, can also be in the form of a lyophilizate and can be made into a solution before parenteral administration by the addition of suitable solvents.

Compositions for inhalation can be administered in aerosol form, as sprays, mist or in form of drops. Aerosols are prepared from solutions or suspensions that can be delivered with a metered-dose inhaler or nebulizer, i.e. a device that delivers a specific amount of medication to the airways or lungs using a suitable propellant, e.g. dichlorodifluoro-methane, trichlorofluoromethane, dichlorotetrafluoroethane, carbon dioxide or other suitable gas, in the form of a short burst of aerosolized medicine that is inhaled by the patient. It is also possible to provide powder sprays for inhalation with a suitable powder base such as lactose or starch.

Eye drops are preferably isotonic aqueous solutions of the active ingredient comprising suitable agents to render the composition isotonic with lacrimal fluid (295-305 mOsm/l). Agents considered are sodium chloride, citric acid, glycerol, sorbitol, mannitol, ethylene glycol, propylene glycol, dextrose, and the like. Furthermore the composition comprise buffering agents, for example phosphate buffer, phosphate-citrate buffer, or Tris buffer (tris(hydroxymethyl)-aminomethane) in order to maintain the pH between 5 and 8, preferably 7.0 to 7.4. The compositions may further contain antimicrobial preservatives, for example parabens, quaternary ammonium salts, such as benzalkonium chloride, polyhexamethylene biguanidine (PHMB) and the like. The eye drops may further contain xanthan gum to produce gel-like eye drops, and/or other viscosity enhancing agents, such as hyaluronic acid, methylcellulose, polyvinylalcohol, or polyvinylpyrrolidone.

### Use of Artificial Transcription Factors in a Method of Treatment

Furthermore the invention relates to artificial transcription factors directed to the OPA1 promoter as described above for use of increasing OPA1 production, and for use in the treatment of diseases influenced by OPA1, in particular for use in the treatment of such eye diseases. Diseases modulated by OPA1 are autosomal dominant optic atrophy, autosomal dominant optic atrophy plus, as wells as normal tension glaucoma.

Likewise the invention relates to a method of treating a disease influenced by OPA1 comprising administering a therapeutically effective amount of an artificial transcription factor of the invention to a patient in need thereof. In particular the invention relates to a method of treating neurodegeneration associated with normal tension glaucoma or dominant optic atrophy. The effective amount of an artificial transcription factor of the invention depends upon the particular type of disease to be treated and upon the species, its age, weight, and individual condition, the individual pharmacokinetic data, and the mode of administration. For administration into the eye, a monthly vitreous injection of 0.5 to 1 mg is preferred. For systemic application, a monthly injection of 10 mg/kg is preferred. In addition, implantation of slow release deposits into the vitreous of the eye is also preferred.

### Use of Artificial Transcription Factors in Animals

Furthermore the invention relates to the use of artificial transcription factors targeting animal OPA1 promoters, to enhance gene product generation. Preferably, the artificial transcription factors are directly applied in suitable compositions for topical applications to animals in need thereof.

### EXAMPLES

### Cloning of DNA Plasmids

For all cloning steps, restriction endonucleases and T4 DNA ligase are purchased from New England Biolabs. Shrimp Alkaline Phosphatase (SAP) is from Promega. The high-fidelity Platinum Pfx DNA polymerase (Invitrogen) is applied in all standard PCR reactions.

DNA fragments and plasmids are isolated according to the manufacturer's instructions using NucleoSpin Gel and PCR Clean-up kit, NucleoSpin Plasmid kit, or NucleoBond Xtra Midi Plus kit (Macherey-Nagel). Oligonucleotides are purchased from Sigma-Aldrich. All relevant DNA sequences of newly generated plasmids were verified by sequencing (Microsynth).

### Cloning of Hexameric Zinc Finger Protein Libraries for Yeast One Hybrid

Hexameric zinc finger protein libraries containing GNN and/or CNN and/or ANN binding zinc finger (ZF) modules are cloned according to Gonzalez B. et al., 2010, Nat Protoc 5, 791-810 with the following improvements. DNA sequences coding for GNN, CNN and ANN ZF modules were synthesized and inserted into pUC57 (GenScript) resulting in pAN1049 (SEQ ID NO: 64), pAN1073 (SEQ ID NO: 65) and pAN1670 (SEQ ID NO: 66), respectively. Stepwise assembly of zinc finger protein (ZFP) libraries is done in pBluescript SK (+) vector. To avoid insertion of multiple ZF modules during each individual cloning step leading to non-functional proteins, pBluescript (and its derived products containing 1ZFP, 2ZFPs, or 3ZFPs) and pAN1049, pAN1073 or pAN1670 are first incubated with one restriction enzyme and afterwards treated with SAP. Enzymes are removed using NucleoSpin Gel and PCR Clean-up kit before the second restriction endonuclease is added.

Cloning of pBluescript-1ZFPL is done by treating 5 μg pBluescript with XhoI, SAP and subsequently SpeI. Inserts are generated by incubating 10 μg pAN1049 (release of 16 different GNN ZF modules) or pAN1073 (release of 15 different CNN ZF modules) or pAN1670 (release of 15 different ANN ZF modules) with SpeI, SAP and subsequently XhoI. For generation of pBluescript-2ZFPL and pBluescript-3ZFPL, 7 μg pBluescript-1ZFPL or pBluescript-2ZFPL are cut with AgeI, dephosphorylated, and cut with SpeI. Inserts are obtained by applying SpeI, SAP, and subsequently XmaI to 10 μg pAN1049 or pAN1073 or pAN1670, respectively. Cloning of pBluescript-6ZFPL was done by treating 14 μg of pBluescript-3ZFPL with AgeI, SAP, and thereafter SpeI to obtain cut vectors. 3ZFPL inserts were released from 20 μg of pBluescript-3ZFPL by incubating with SpeI, SAP, and subsequently XmaI.

Ligation reactions for libraries containing one, two, and three ZFPs were set up in a 3:1 molar ratio of insert:vector using 200 ng cut vector, 400 U T4 DNA ligase in 20 μl total volume at RT (room temperature) overnight. Ligation reactions of hexameric zinc finger protein libraries included 2000 ng pBluescript-3ZFPL, 500 ng 3ZFPL insert, 4000 U T4 DNA ligase in 200 μl total volume, which were divided into ten times 20 μl and incubated separately at RT overnight. Portions of ligation reactions were transformed into Escherichia coli by several methods depending on the number of clones required for each library. For generation of pBluescript-1ZFPL and pBluescript-2ZFPL, 3 μl of ligation reaction were directly used for heat shock transformation of E. coli NEB 5-alpha. Plasmid DNA of ligation reactions of pBluescript-3ZFPL was purified using NucleoSpin Gel and PCR Clean-up kit and transformed into electrocompetent E. coli NEB 5-alpha (EasyjecT Plus electroporator from EquiBio or Multiporator from Eppendorf, 2.5 kV and 25 μF, 2 mm electroporation cuvettes from Bio-Rad). Ligation reactions of pBluescript-6ZFP libraries were applied to NucleoSpin Gel and PCR Clean-up kit and DNA was eluted in 15 μl of deionized water. About 60 ng of desalted DNA were mixed with 50 μl NEB 10-beta electrocompetent E. coli (New England Biolabs) and electroporation was performed as recommended by the manufacturer using EasyjecT Plus or Multiporator, 2.5 kV, 25 μF and 2 mm electroporation cuvettes. Multiple electroporations were performed for each library and cells were directly pooled afterwards to increase library size. After heat shock transformation or electroporation, SOC medium was applied to the bacteria and after 1 h of incubation at 37° C. and 250 rpm, 30 μl of SOC culture were used for serial dilutions and plating on LB plates containing ampicillin. The next day, total number of obtained library clones was determined. In addition, ten clones of each library were chosen to isolate plasmid DNA and to check incorporation of inserts by restriction enzyme digestion. At least three of these plasmids were sequenced to verify diversity of the library. The remaining SOC culture was transferred to 100 ml LB medium containing ampicillin and cultured overnight at 37° C. and 250 rpm. Those cells were used to prepare plasmid Midi DNA for each library.

For yeast one hybrid screens, hexameric zinc finger protein libraries are transferred to a compatible prey vector. For that purpose, the multiple cloning site of pGAD10 (Clontech) was modified by cutting the vector with XhoI/EcoRI and inserting annealed oligonucleotides OAN971 (TCGACAGGCCCAGGCGGCCCTCGAGGATATCATGATG ACTAGTGGCCAGGCCGGCCC, SEQ ID NO: 67) and OAN972 (AATTGGGCCGGC CTGGCCACTAGTCATCATGATATCCTCGAGGGCCGCCTGGGCCTG, SEQ ID NO: 68). The resulting vector pAN1025 (SEQ ID NO: 69) was cut and dephosphorylated, 6ZFP library inserts were released from pBluescript-6ZFPL by XhoI/SpeI. Ligation reactions and electroporations into NEB 10-beta electrocompetent E. coli were done as described above for pBluescript-6ZFP libraries.

For improved yeast one hybrid screening, hexameric zinc finger libraries are also transferred into an improved prey vector pAN1375 (SEQ ID NO: 70). This prey vector was constructed as follows: pRS315 (SEQ ID NO: 71) was cut ApaI/NarI and annealed OAN1143 (CGCCGCATGCATTCATGCAGGCC, SEQ ID NO: 72) and OAN1144 (TGCATGAATGCATGCGG, SEQ ID NO: 73) were inserted yielding pAN1373 (SEQ ID NO: 74). A SphI insert from pAN1025 was ligated into pAN1373 cut with SphI to obtain pAN1375.

For further improved yeast one hybrid screening, hexameric zinc finger libraries are also transferred into an improved prey vector pAN1920 (SEQ ID NO: 75).

For even further improved yeast one hybrid screening, hexameric zinc finger libraries are inserted into prey vector pAN1992 (SEQ ID NO: 76).

### Cloning of Bait Plasmids for Yeast One Hybrid Screening

For each bait plasmid, a 60 bp sequence containing a potential artificial transcription factor target site of 18 bp in the center is selected and a NcoI site is included for restriction analysis. Oligonucleotides are designed and annealed in such a way to produce 5′ HindIII and 3′ XhoI sites which allowed direct ligation into pAbAi (Clontech) cut with HindIII/XhoI. Digestion of the product with NcoI and sequencing are used to confirm assembly of the bait plasmid.

### Yeast Strain and Media

Saccharomyces cerevisiae Y1H Gold was purchased from Clontech, YPD medium and YPD agar from Carl Roth. Synthetic drop-out (SD) medium contained 20 g/l glucose, 6.8 g/l Na₂HPO₄.2H₂O, 9.7 g/l NaH₂PO₄.2H₂O (all from Carl Roth), 1.4 g/l yeast synthetic drop-out medium supplements, 6.7 g/l yeast nitrogen base, 0.1 g/l L-tryptophan, 0.1 g/l L-leucine, 0.05 g/l L-adenine, 0.05 g/l L-histidine, 0.05 g/l uracil (all from Sigma-Aldrich). SD-U medium contained all components except uracil, SD-L was prepared without L-leucine. SD agar plates did not contain sodium phosphate, but 16 g/l Bacto Agar (BD). Aureobasidin A (AbA) was purchased from Clontech.

### Preparation of Bait Yeast Strains

About 5 μg of each bait plasmid are linearized with BstBI in a total volume of 20 μl and half of the reaction mix is directly used for heat shock transformation of S. cerevisiae Y1H Gold. Yeast cells are used to inoculate 5 ml YPD medium the day before transformation and grown overnight on a roller at RT. One milliliter of this pre-culture is diluted 1:20 with fresh YPD medium and incubated at 30° C., 225 rpm for 2-3 h. For each transformation reaction 1 OD₆₀₀ cells are harvested by centrifugation, yeast cells are washed once with 1 ml sterile water and once with 1 ml TE/LiAc (10 mM Tris/HCl, pH 7.5, 1 mM EDTA, 100 mM lithium acetate). Finally, yeast cells are resuspended in 50 μl TE/LiAc and mixed with 50 μg single stranded DNA from salmon testes (Sigma-Aldrich), 10 μl of BstBI-linearized bait plasmid (see above), and 300 μl PEG/TE/LiAc (10 mM Tris/HCl, pH 7.5, 1 mM EDTA, 100 mM lithium acetate, 50% (w/v) PEG 3350). Cells and DNA are incubated on a roller for 20 min at RT, afterwards placed into a 42° C. water bath for 15 min. Finally, yeast cells are collected by centrifugation, resuspended in 100 μl sterile water and spread onto SD-U agar plates. After 3 days of incubation at 30° C. eight clones growing on SD-U from each transformation reaction are chosen to analyze their sensitivity towards aureobasidin A (AbA). Pre-cultures were grown overnight on a roller at RT. For each culture, OD₆₀₀ was measured and OD₆₀₀=0.3 was adjusted with sterile water. From this first dilution five additional 1/10 dilution steps were prepared with sterile water. For each clone 5 μl from each dilution step were spotted onto agar plates containing SD-U, SD-U 100 ng/ml AbA, SD-U 150 ng/ml AbA, and SD-U 200 ng/ml AbA. After incubation for 3 days at 30° C., three clones growing well on SD-U and being most sensitive to AbA are chosen for further analysis. Stable integration of bait plasmid into yeast genome is verified by Matchmaker Insert Check PCR Mix 1 (Clontech) according to the manufacturer's instructions. One of three clones is used for subsequent Y1H screen.

Transformation of Bait Yeast Strain with Hexameric Zinc Finger Protein Library

About 500 μl of yeast bait strain pre-culture are diluted into 1 l YPD medium and incubated at 30° C. and 225 rpm until OD₆₀₀=1.6-2.0 (circa 20 h). Cells are collected by centrifugation in a swing-out rotor (5 min, 1500×g, 4° C.). Preparation of electrocompetent cells is done according to Benatuil L. et al., 2010, Protein Eng Des Sel 23, 155-159. For each transformation reaction, 400 μl electrocompetent bait yeast cells are mixed with 1 μg prey plasmids encoding 6ZFP libraries and incubated on ice for 3 min. Cell-DNA suspension is transferred to a pre-chilled 2 mm electroporation cuvette. Multiple electroporation reactions (EasyjecT Plus electroporator or Multiporator, 2.5 kV and 25 μF) are performed until all yeast cell suspension has been transformed. After electroporation yeast cells are transferred to 100 ml of 1:1 mix of YPD:1 M Sorbitol and incubated at 30° C. and 225 rpm for 60 min. Cells are collected by centrifugation and resuspended in 1-2 ml of SD-L medium. Aliquots of 200 μl are spread on 15 cm SD-L agar plates containing 1000-4000 ng/ml AbA. In addition, 50 μl of cell suspension are used to make 1/100 and 1/1000 dilutions and 50 μl of undiluted and diluted cells are plated on SD-L. All plates are incubated at 30° C. for 3 days. The total number of obtained clones is calculated from plates with diluted transformants. While SD-L plates with undiluted cells indicate growth of all transformants, AbA-containing SD-L plates only resulted in colony formation if the prey 6ZFP bound to its bait target site successfully.

### Verification of Positive Interactions and Recovery of 6ZFP-Encoding Prey Plasmids

For initial analysis, forty good-sized colonies are picked from SD-L plates containing the highest AbA concentration and yeast cells were restreaked twice on SD-L with 1000-4000 ng/ml AbA to obtain single colonies. For each clone, one colony is used to inoculate 5 ml SD-L medium and cells are grown at RT overnight. The next day, OD₆₀₀=0.3 is adjusted with sterile water, five additional 1/10 dilutions are prepared and 5 μl of each dilution step are spotted onto SD-L, SD-L 500 ng/ml AbA, 1000 ng/ml AbA, SD-L 1500 ng/ml AbA, SD-L 2000 ng/ml AbA, SD-L 2500 ng/ml AbA, SD-L 3000 ng/ml AbA, and SD-L 4000 ng/ml AbA plates. Clones are ranked according to their ability to grow on high AbA concentration. From best growing clones 5 ml of initial SD-L pre-culture are used to spin down cells and to resuspend them in 100 μl water or residual medium. After addition of 50 U lyticase (Sigma-Aldrich, L2524) cells are incubated for several hours at 37° C. and 300 rpm on a horizontal shaker. Generated spheroblasts are lysed by adding 10 μl 20% (w/v) SDS solution, mixed vigorously by vortexing for 1 min and frozen at −20° C. for at least 1 h. Afterwards, 250 μl A1 buffer from NucleoSpin Plasmid kit and one spatula tip of glass beads (Sigma-Aldrich, G8772) are added and tubes are mixed vigorously by vortexing for 1 min. Plasmid isolation is further improved by adding 250 μl A2 buffer from NucleoSpin Plasmid kit and incubating for at least 15 min at RT before continuing with the standard NucleoSpin Plasmid kit protocol. After elution with 30 μl of elution buffer 5 μl of plasmid DNA are transformed into E. coli DH5 alpha by heat shock transformation. Two individual colonies are picked from ampicillin-containing LB plates, plasmids are isolated and library inserts are sequenced. Obtained results are analyzed for consensus sequences among the 6ZFPs for each target site.

### Cloning of OPA1 Gene Promoter Region for Combined Secreted Luciferase and Alkaline Phosphatase Assay

A DNA fragment containing the OPA1 promoter region was cloned into pAN1485 (NEG-PG04, GeneCopeia) resulting in reporter plasmid pAN1680 (SEQ ID NO: 77) containing secreted Gaussia luciferase under the control of the OPA1 gene promoter and secreted embryonic alkaline phosphatase under the control of the constitutive CMV promoter allowing for normalization of luciferase to alkaline phosphatase signal.

### Cloning of Artificial Transcription Factors for Mammalian Transfection

DNA fragments encoding polydactyl zinc finger proteins either generated through Gensynthesis (GenScript) or selected by yeast one hybrid are cloned using standard procedures with AgeI/XhoI into mammalian expression vectors for expression in mammalian cells as fusion proteins between the zinc finger array of interest, a SV40 NLS, a 3×myc epitope tag and a N-terminal KRAB domain (pAN1255—SEQ ID NO: 78), a C-terminal KRAB domain (pAN1258—SEQ ID NO: 79), a SID domain (pAN1257—SEQ ID NO: 80) or a VP64 activating domain (pAN1510—SEQ ID NO: 81).

Plasmids for the generation of stably transfected, tetracycline-inducible cells were generated as follows: DNA fragments encoding artificial transcriptions factors comprising polydactyl zinc finger domain, a regulatory domain (N-terminal KRAB, C-terminal KRAB, SID or VP64), SV40 NLS and a 3×myc epitope tag are cloned into pcDNA5/FRT/TO (Invitrogen) using EcoRV/NotI.

### Cell Culture and Transfections

HeLa cells are grown in Dulbecco's Modified Eagle's Medium (DMEM) supplemented with 4.5 g/l glucose, 10% heat-inactivated fetal bovine serum, 2 mM L-glutamine, and 1 mM sodium pyruvate (all from Sigma-Aldrich) in 5% CO₂ at 37° C. For luciferase reporter assay, 7000 HeLa cells/well are seeded into 96 well plates. Next day, co-transfections are performed using Effectene Transfection Reagent (Qiagen) according to the manufacturer's instructions. Plasmid midi preparations coding for artificial transcription factor and for luciferase are used in the ratio 3:1. Medium is replaced by 100 μl per well of fresh DMEM 6 h and 24 h after transfection.

### Generation and Maintenance of Flp-Ln™ T-Rex™ 293 Expression Cell Lines

Stable, tetracycline inducible Flp-ln™ T-Rex™ 293 expression cell lines are generated by Flp Recombinase-mediated integration. Using Flp-ln™ T-Rex™ Core Kit, the Flp-ln™ T-Rex™ host cell line is generated by transfecting pFRT/lacZeo target site vector and pcDNA6/TR vector. For generation of inducible 293 expression cell lines, the pcDNA5/FRT/TO expression vector containing the gene of interest is integrated via Flp recombinase-mediated DNA recombination at the FRT site in the Flp-ln™ T-Rex™ host cell line. Stable Flp-ln™ T-Rex™ expression cell lines are maintained in selection medium containing (DMEM; 10% Tet-FBS; 2 mM glutamine; 15 μg/ml blasticidine and 100 μg/ml hygromycin). For induction of gene expression tetracycline is added to a final concentration of 1 μg/ml.

### Combined Luciferase/SEAP Promoter Activity Assay

HeLa cells are co-transfected with an artificial transcription factor expression construct and a plasmid carrying secreted Gaussia luciferase under the control of the OPA1 promoter and secreted alkaline phosphatase under the control of the constitutive CMV promoter (Gaussia luciferase Glow Assay Kit, Pierce; SEAP Reporter Gene Assay chemiluminescent, Roche). Two days following transfection, cell culture supernatants were collected and luciferase activity and SEAP activity were measured using Gaussia Luciferase Glow Assay Kit (Thermo Scientific) and the SEAP reporter gene assay (Roche), respectively. Co-transfection of an expression plasmid for an inactive artificial transcription factor with all cysteine residues in the zinc finger domain exchanged to serine residues served as control. Luciferase activity was normalized to SEAP activity and expressed as percentage of control.

### Determination of Gene Expression Levels by Quantitative RT-PCR

Total RNA is isolated from cells using the RNeasy Plus Mini Kit (Qiagen, Hilden, Germany) according to the manufacturer's instructions. Frozen cell pellets are resuspended in RLT Plus Lysis buffer containing 10 μl/ml R-mercaptoethanol. After homogenization using QIAshredder spin columns, total lysate is transferred to gDNA Eliminator spin columns to eliminate genomic DNA. One volume of 70% ethanol is added and total lysate is transferred to RNeasy spin columns. After several washing steps, RNA is eluted in a final volume of 30 μl RNase free water. RNA is stored at −80° C. until further use. Synthesis of cDNA is performed using the High Capacity cDNA Reverse

Transcription Kit (Applied Biosystems, Branchburg, N.J., USA) according to the manufacturer's instructions. cDNA synthesis is carried out in 20 μl of total reaction volume containing 2 μl 10× Buffer, 0.8 μl 25×dNTP Mix, 2 μl 10×RT Random Primers, 1 μl Multiscribe Reverse Transcriptase and 4.2 μl H₂O. A final volume of 10 μl RNA is added and the reaction is performed under the following conditions: 10 minutes at 25° C., followed by 2 hours at 37° C. and a final step of 5 minutes at 85° C. Quantitative PCR is carried out in 20 μl of total reaction volume containing 1 μl 20× TaqMan Gene Expression Master Mix, 10.0 μl TaqMan® Universal PCR Master Mix (both Applied Biosystems, Branchburg, N.J., USA) and 8 μl H₂O. For each reaction 1 μl of cDNA is added. qPCR is performed using the ABI PRISM 7000 Sequence Detection System (Applied Biosystems, Branchburg, N.J., USA) under the following conditions: an initiation step for 2 minutes at 50° C. is followed by a first denaturation for 10 minutes at 95° C. and a further step consisting of 40 cycles of 15 seconds at 95° C. and 1 minute at 60° C.

### Cloning of Artificial Transcription Factors for Bacterial Expression

DNA fragments encoding artificial transcription factors are cloned using standard procedures with EcoRV/NotI into bacterial expression vector pAN983 (SEQ ID NO: 82) based on pET41a+ (Novagen) for expression in E. coli as His₆-tagged fusion proteins between the artificial transcription factor and the TAT protein transduction domain.

Expression constructs for the bacterial production of transducible artificial transcription factors in suitable E. coli host cells such as BL21(DE3) targeting OPA1 are pAN1964 (SEQ ID NO: 83), pAN2053 (SEQ ID NO: 84), pAN2055 (SEQ ID NO: 85), pAN2057 (SEQ ID NO: 86), pAN2059 (SEQ ID NO: 87), pAN2061 (SEQ ID NO: 88), and pAN2063 (SEQ ID NO: 89).

### Production of Artificial Transcription Factor Protein

E. coli BL21(DE3) transformed with expression plasmid for a given artificial transcription factor were grown in 1 l LB media supplemented with 100 μM ZnCl₂ until OD₆₀₀ between 0.8 and 1 was reached, and induced with 1 mM IPTG for two hours. Bacteria were harvested by centrifugation, bacterial lysate was prepared by sonication, and inclusion bodies were purified. To this end, inclusion bodies were collected by centrifugation (5000 g, 4° C., 15 minutes) and washed three times in 20 ml of binding buffer (50 mM HEPES, 500 mM NaCl, 10 mM imidazole; pH 7.5). Purified inclusion bodies were solubilized on ice for one hour in 30 ml of binding buffer A (50 mM HEPES, 500 mM NaCl, 10 mM imidazole, 6 M GuHCl; pH 7.5). Solubilized inclusion bodies were centrifuged for 40 minutes at 4° C. and 13,000 g and filtered through 0.45 μm PVDF filter. His-tagged artificial transcription factors were purified using His-Trap columns on an Äktaprime FPLC (GEHealthcare) using binding buffer A and elution buffer B (50 mM HEPES, 500 mM NaCl, 500 mM imidazole, 6 M GuHCl; pH 7.5). Fractions containing purified artificial transcription factor were pooled and dialyzed at 4° C. overnight against buffer S (50 mM Tris-HCl, 500 mM NaCl, 200 mM arginine, 100 μM ZnCl₂, 5 mM GSH, 0.5 mM GSSG, 50% glycerol; pH 7.5) in case the artificial transcription factor contained a SID domain, or against buffer K (50 mM Tris-HCl, 300 mM NaCl, 500 mM arginine, 100 μM ZnCl₂, 5 mM GSH, 0.5 mM GSSG, 50% glycerol; pH 8.5) for KRAB domain containing artificial transcription factors. Following dialysis, protein samples were centrifuged at 14,000 rpm for 30 minutes at 4° C. and sterile filtered using 0.22 μm Millex-GV filter tips (Millipore). For artificial transcription factors containing VP64 activation domain, the protein was produced from the soluble fraction (binding buffer: 50 mM NaPO₄ pH 7.5, 500 mM NaCl, 10 mM imidazole; elution buffer 50 mM HEPES pH 7.5, 500 mM NaCl, 500 mM imidazole) using His-Bond Ni-NTA resin (Novagen) according to manufactures recommendation. Protein was dialyzed against VP64-buffer (550 mM NaCl pH 7.4, 400 mM arginine, 100 μM ZnCl₂).

### Determination of DNA Binding Activity of Artificial Transcription Factors Using ELDIA (Enzyme-Linked DNA Interaction Assay)

BSA pre-blocked nickel coated plates (Pierce) are washed 3 times with wash buffer (25 mM Tris/HCl pH 7.5, 150 mM NaCl, 0.1% BSA, 0.05% Tween-20). Plates are coated with purified artificial transcription factor under saturating conditions (50 pmol/well) in storage buffer and incubated 1 h at RT with slight shake. After 3 washing steps, 1×10⁻¹² to 5×10⁻⁷ M of annealed, biotinylated oligos containing 60 bp promoter sequence are incubated in binding buffer (10 mM Tris/HCl pH 7.5, 60 mM KCl, 1 mM DTT, 2% glycerol, 5 mM MgCl₂ and 100 μM ZnCl₂) in the presence of unspecific competitor (0.1 mg/ml ssDNA from salmon sperm, Sigma) with the bound artificial transcription factor for 1 h at RT. After washing (5 times), wells are blocked with 3% BSA for 30 minutes at RT. Anti-streptavidin-HRP is added in binding buffer for 1 h at RT. After 5 washing steps, TMB substrate (Sigma) is added and incubated for 2 to 30 minutes at RT. Reaction is stopped by addition of TMB stop solution (Sigma) and sample extinction is read at 450 nm. Data analysis of ligand binding kinetics is done using Sigma Plot V8.1 according to Hill.

### Protein Transduction

Cells grown to about 80% confluency are treated with 0.01 to 1 μM artificial transcription factor or mock treated for 2 h to 120 h with optional addition of artificial transcription factor every 24 h in OptiMEM or growth media at 37° C. Optionally, 10-500 μM ZnCl₂ are added to the growth media. For immunofluorescence, cells are washed once in PBS, trypsinized and seeded onto glass cover slips for further examination.

### Immunofluorescence

Cells are fixed with 4% paraformaldehyde, treated with 0.15% Triton X-100 for 15 minutes, blocked with 10% BSAPBS and incubated overnight with mouse anti-HA antibody (1:500, H9658, Sigma) or mouse anti-myc (1:500, M5546, Sigma). Samples are washed three times with PBS/1% BSA, and incubated with goat anti-mouse antibodies coupled to Alexa Fluor 546 (1:1000, Invitrogen) and counterstained using DAPI (1:1000 of 1 mg/ml for 3 minutes, Sigma). Samples are analyzed using fluorescence microscopy.

### Western Blotting

For measuring protein levels, cells are lysed using RIPA buffer (Pierce) and protein lysates are mixed with Laemmli sample buffer. Proteins are separated by SDS-PAGE according to their size and transferred using electroblotting to nitrocellulose membranes. Detection of proteins is performed using specific primary antibodies raised in mice or rabbits. Detection of primary antibodies is performed either by secondary antibodies coupled to horseradish peroxidase and luminescence-based detection (ECL plus, Pierce) or secondary antibodies coupled to DyLight700 or DyLight800 fluorescent detected and quantified using a infrared laser scanner.

### Measuring Mitochondrial Function

For flow cytometric analysis, treated cells are harvested with 10 mM EDTA/PBS. Mock treated cells are used as control. For measuring mitochondrial membrane potential, cells are resuspended in FACS buffer P (PBS, 5 mM EDTA, 0.5% (w/v) BSA, 1 μg/ml 4′,6-diamidino-2-phenylindole dihydrochloride (DAPI, Sigma), 10 nM tetramethylrhodamine ethylester (TMRE, Sigma)) and incubated for 30 min at 37° C. prior to analysis. Treatment with 50 μM carbonyl cyanide 3-chlorophenylhydrazone (CCCP, Sigma) to dissipate mitochondrial membrane potential serves as control. For measurement of mitochondrial mass, cells are resuspended in FACS buffer M (PBS, 5 mM EDTA, 0.5% (w/v) BSA, 1 μg/ml DAPI and 100 nM MitoTracker green FM (Invitrogen)) and incubated for 30 min at 37° C. prior to analysis. For mitochondrial ROS measurements, cells are resuspended in FACS buffer R (PBS, 5 mM EDTA, 0.5% BSA, 1 μg/ml DAPI and 5 μM MitoSOX (Invitrogen), incubated for 10 min at 37° C., washed with PBS, and resuspended in FACS buffer R2 (PBS, 5 mM EDTA, 0.5% (w/v) BSA). Flow cytometric analysis is performed on a CyAn_{ADP} (Dako) using FlowJo software (Tree Star Inc.).

### Measuring Apoptotic Induction

Cells are fixed for 30 minutes at RT with 4% EM-grade paraformaldehyde (Pierce, 28908) in phosphate-buffered saline (PBS). Then, cells are permeabilized with 0.15% (v/v) Triton X-100 in PBS for 15 min at RT, followed by blocking with 10% (w/v) BSA in PBS for 1 hour at RT. Samples are incubated overnight at 4° C. with mouse anti-cytochrome c antibodies (BD Biosciences, 556432, 1:1000) diluted in blocking buffer. Cells are washed three times for 15 minutes with blocking buffer and then incubated for 1 hour at RT with Alexa Fluor 546-conjugated goat anti-mouse IgG antibodies (Invitrogen). Cytochrome c release as measure of apoptosis is analyzed by fluorescence microscopy by a blinded observer. Mock treated cells serve as control.

1. An artificial transcription factor comprising a polydactyl zinc finger protein targeting specifically the OPA1 gene promoter fused to an activatory protein domain and a nuclear localization sequence.

2. The artificial transcription factor according to claim 1 further comprising a protein transduction domain.

3. The artificial transcription factor according to claim 1 comprising a hexameric zinc finger protein.

4. The artificial transcription factor according to claim 1, wherein the activatory protein domain is VP16 of SEQ ID NO: 1, VP64 of SEQ ID NO: 2, CJ7 of SEQ ID NO: 3, p65TA1 of SEQ ID NO: 4, SAD of SEQ ID NO: 5, NF-1 of SEQ ID NO: 6, AP-2 of SEQ ID NO: 7, SP1-A of SEQ ID NO: 8, SP1-B of SEQ ID NO: 9, Oct-1 of SEQ ID NO: 10, Oct-2 of SEQ ID NO: 11, Oct2-5× of SEQ ID NO: 12, MTF-1 of SEQ ID NO: 13, BTEB-2 of SEQ ID NO: 14 or LKLF of SEQ ID NO: 15.

5. The artificial transcription factor according to claim 1, wherein the nuclear localization sequences is a cluster of basic amino acids containing the K-K/R-X-K/R consensus sequence or the SV40 NLS of SEQ ID NO: 62.

6. The artificial transcription factor according to claim 2, wherein the protein transduction domain is the HIV derived TAT peptide of SEQ ID NO: 16, the synthetic peptide mT02 of SEQ ID NO: 18, the synthetic peptide mT03 of SEQ ID NO: 19, the R9 peptide of SEQ ID NO: 20, or the ANTP domain of SEQ ID NO: 21.

7. The artificial transcription factor according to claim 1 comprising a zinc finger protein of a protein sequence selected from the group consisting of SEQ ID NO: 26 to 43.

8. The artificial transcription factor according to claim 1 further comprising a polyethylene glycol residue.

9. A pharmaceutical composition comprising the artificial transcription factor according to claim 1.

10. A nucleic acid coding for an artificial transcription factor according to claim 1.

11. A vector comprising the nucleic acid according to claim 10.

12. The vector of claim 11, which is a viral vector.

13. A host cell comprising the vector according to claim 11.

14. An E. coli host cell according to claim 13 containing an expression construct of SEQ ID NO: 83 to 89.

15. A viral carrier comprising the nucleic acid according to claim 10.

16. The viral carrier of claim 15, which is selected from the group consisting of adeno-associated viruses, retroviruses, lentiviruses, adenoviruses, pseudotyped adeno-associated viruses, pseudotyped retroviruses, pseudotyped lentiviruses and pseudotyped adenoviruses.

17. A pharmaceutical composition comprising the viral carrier according to claim 15.

18. The artificial transcription factor according to claim 1 for use in increasing expression from the OPA1 gene promoter.

19. The nucleic acid according to claim 10 for use in increasing expression from the OPA1 gene promoter.

20. The artificial transcription factor according to claim 1 for use in treating autosomal dominant atrophy, autosomal dominant atrophy plus and glaucoma.

21. The nucleic acid according to claim 10 for use in treating autosomal dominant atrophy, autosomal dominant atrophy plus and glaucoma.

22. A method of treatment of autosomal dominant atrophy, autosomal dominant atrophy plus or glaucoma comprising administering a therapeutically effective amount of an artificial transcription factor according to claim 1 or a nucleic acid coding for an artificial transcription factor according to claim 1 to a patient in need thereof.
